(19) United States
(12) Patent Application Publication
(10) Pub. No.: US 2026/0000332 A1
(43) Pub. Date: Jan. 1, 2026

(54) NEURAL INTERFACE FIBER

(71) Applicant: NeuroBionics Inc., Somerville, MA (US)

(72) Inventors: Marc-Joseph Antonini, Somerville, MA (US); Nicolette L. Driscoll, Somerville, MA (US); Erin B. Rousseau, Somerville, MA (US)

(21) Appl. No.: 19/251,488

(22) Filed: Jun. 26, 2025

Related U.S. Application Data
(60) Provisional application No. 63/664,401, filed on Jun. 26, 2024.

(51) Int. Cl. A61B 5/268; A61B 5/00; A61N 1/05

(52) U.S. Cl. CPC A61B 5/268; A61B 5/6876; A61N 1/05; A61B 2560/0468; A61B 2560/063; A61B 2562/063; A61B 2562/227

(57) ABSTRACT

Disclosed are examples of a neural interface fiber. The neural interface fiber includes a plurality of conductive structures, a cladding adapted to substantially encompass the plurality of conductive structures, where the cladding is a dielectric insulation, where the cladding includes one or more openings exposing a portion of a respective conductive structure of the plurality of conductive structures within the cladding enabling the exposed portion to contact tissue external to the cladding at the one or more openings. In another example, an end cap may be fastened to a distal end of the cladding and configured to electrically insulate the plurality of conductive structures, where the plurality of conductive structures are configured to connect to an electrical signaling/recording device.

### CROSS-REFERENCE TO RELATED APPLICATIONS

This application claims priority to U.S. Provisional Patent Application No. 63/664,401 filed on Jun. 26, 2024, the entire contents of which are hereby incorporated by reference.

### BACKGROUND

Neuromodulation is a therapeutic approach that involves altering neurological activity-nerves or neurons-through targeted delivery of electrical stimulation or chemical agents to specific neurological sites in the body. This technique is used to manage various chronic pain conditions and neurological disorders.

Neuromodulation therapies are often considered when conventional treatments fail. They offer a promising alternative by directly influencing the nervous system's activity, providing relief from chronic pain and improving patients' overall well-being. Neuromodulation today involves advanced techniques to manage chronic pain and neurological disorders.

There are different types of neuromodulation, such as brain neuromodulation, spinal cord neuromodulation, and peripheral nerve neuromodulation.

Deep brain stimulation (DBS) is a prominent method for brain neuromodulation and is a method in which electrodes are implanted in specific brain regions. These electrodes deliver electrical impulses to modulate abnormal neural activity. DBS is commonly used to treat movement disorders like Parkinson's disease, essential tremor, and dystonia. It is also being explored for psychiatric conditions such as depression and obsessive-compulsive disorder.

Meanwhile, spinal cord stimulation (SCS) involves placing electrodes in the epidural space of the spinal cord. These electrodes generate electrical pulses that interfere with pain signals before they reach the brain. SCS is effective for conditions like chronic back pain, failed back surgery syndrome, and complex regional pain syndrome. The goal is to reduce pain perception and improve quality of life.

Peripheral nerve stimulation (PNS) is used to target specific peripheral nerves outside the brain and spinal cord. Electrodes are placed near the affected nerves to deliver electrical impulses, which can alleviate pain and improve function. PNS is used for conditions such as peripheral neuropathy, post-surgical pain, and migraines. This method is less invasive and can be tailored to target localized pain areas.

However, each therapy, DBS, SCS, and PNS, have risks and possible adverse effects, such as surgical risks, device-related issues and/or neurological effects. Some potential risks associated with the implantation procedures may include infection, bleeding, and device malfunction. Hardware complications, such as lead migration or breakage, can also occur. Neurological effects for some patients may include experiencing changes in sensation, muscle weakness, or unintended stimulation of other nerves.

While neuromodulation offers significant benefits, it is crucial for patients to discuss potential risks with their healthcare providers to make informed decisions.

In addition to DBS, SCS and PNS, there are several other types of neurostimulators available, but each comes with unique applications and limitations. For example, a vagus nerve stimulator (VNS) is used primarily for epilepsy and depression, but can cause side effects such as voice changes, throat pain, and breathing difficulties. And another neurostimulator may be the Inspire V, which is a next-generation device for obstructive sleep apnea that reduces surgical complexity and time. However, it may still face challenges like device compatibility and patient adaptation.

Despite their benefits, neurostimulators can have limitations such as the above-described surgical risks, device malfunctions, and varying patient responses. Continuous advancements aim to mitigate these issues and improve patient outcomes.

There is a need for less invasive methods to deliver neuromodulation therapies to the target neural tissue.

### BRIEF SUMMARY

In one aspect, a neural interface fiber is provided that includes a plurality of conductive structures, a cladding adapted to substantially encompass the plurality of conductive structures, where the cladding is a dielectric insulation and includes one or more openings exposing a portion of a respective conductive structure of the plurality of conductive structures within the cladding enabling the exposed portion to contact tissue external to the cladding at the one or more openings, and an end cap fastened to a distal end of the cladding and configured to electrically insulate the plurality of conductive structures, where the plurality of conductive structures are configured to connect to an electrical signaling/recording device.

In one aspect, a neural interface fiber is provided that includes a plurality of conductive structures, and a cladding adapted to insulate the plurality of the conductive structures, where the exposed portions of respective conductive structures of the plurality of conductive structures in the cladding are electrodes operable to contact tissue external to the cladding. The neural interface fiber also includes an end cap fastened to a distal end of the cladding and configured to electrically insulate the plurality of conductive structures at the distal end.

### BRIEF DESCRIPTION OF THE DRAWINGS

To easily identify the discussion of any particular element or act, the most significant digit or digits in a reference number refer to the figure number in which that element is first introduced.

FIG. 1 shows a perspective view of a section of an exemplary neural interface fiber according to some embodiments.

FIG. 2 illustrates an exemplary configuration of a neural interface fiber according to the disclosed subject matter.

FIG. 3 illustrates another exemplary configuration of a neural interface fiber according to the disclosed subject matter.

FIG. 4 illustrates a magnified view of an exemplary opening in cladding of a neural interface fiber to expose a portion of the conductive structure within in accordance with the disclosed subject matter.

FIG. 5 illustrates a portion of an exemplary neural interface fiber with a pattern of electrodes according to the disclosed subject matter.

FIG. 6 illustrates a portion of an exemplary helical structure of an exemplary neural interface fiber according to the disclosed subject matter.

FIG. 7 illustrates a portion of an exemplary neural interface fiber according to the disclosed subject matter.

FIG. 8 illustrates an exemplary end cap with an exemplary delivery feature according to the disclosed subject matter.

FIG. 9 illustrates a detailed view of an exemplary end cap with an exemplary delivery feature according to the disclosed subject matter.

FIG. 10A illustrates a portion of another exemplary helical structure of an exemplary neural interface fiber according to the disclosed subject matter.

FIG. 10B illustrates an exemplary modified helical structure in FIG. 10A according to the disclosed subject matter.

FIG. 10C illustrates a bottom plan view of an exemplary portion of the exemplary helical structure of the example of FIG. 10A.

FIG. 11 illustrates a block diagram of neuromodulation system utilizing the exemplary neural interface fibers of the disclosed subject matter.

FIG. 12 is a graphic illustration of an exemplary implementation of the disclosed exemplary neural interface fibers.

FIG. 13 is another graphic illustration of an exemplary implementation of the disclosed exemplary neural interface fibers.

FIG. 14 illustrates an exemplary arrangement for the formation of a neural interface fiber according to the disclosed subject matter.

FIG. 15A illustrates a perspective view of an exemplary system component usable in a larger system to form an exemplary helical structure of an exemplary neural interface fiber according to the disclosed subject matter.

FIG. 15B illustrates a side view of an exemplary system component usable in a larger system to form an exemplary helical structure of an exemplary neural interface fiber according to the disclosed subject matter.

FIG. 15C illustrates a perspective view of an exemplary helical structure of an exemplary neural interface fiber disengaged from an exemplary system component after formation of the helical structure according to the disclosed subject matter.

### DETAILED DESCRIPTION

A number of less invasive devices and methods to deliver neuromodulation therapies to targeted neural tissue are described herein. These less invasive devices and methods may provide a neural interface fiber having electrodes operable to utilize blood vessels as natural conduits to the brain and the peripheral nerves and offer a potential access point for delivering neuromodulation therapies across the body.

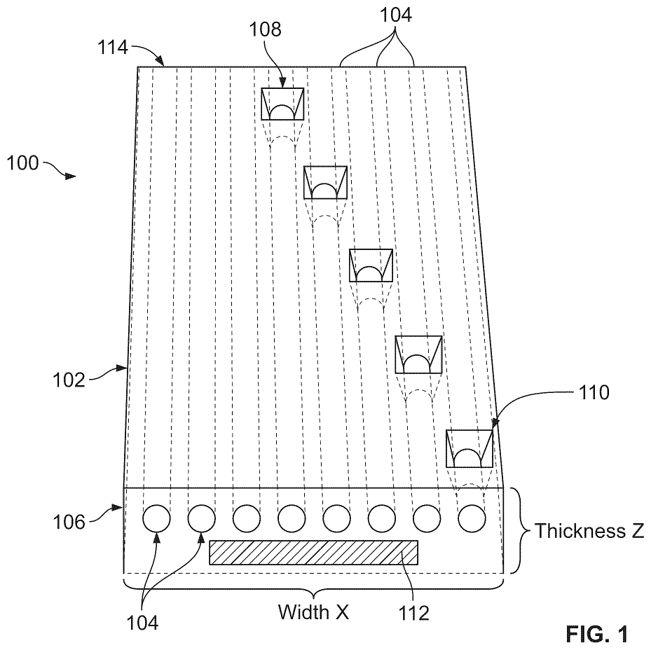

Reference is now made to FIG. 1, which shows a perspective view of a section of a neural interface fiber 100 according to some embodiments.

The neural interface fiber 100 includes a cladding 102 and a plurality of conductive structures 104. The cladding 102 may be adapted to substantially encompass the plurality of conductive structures 104. The cladding 102 may be a dielectric insulation or the like and is a flexible material that allows the neural interface fiber 100 to bend and stretch in anatomical structures, such as blood vessels. The cladding 102 may include one or more openings 108 exposing a portion of a respective conductive structure. The exposed portion of the respective conductive structure may be referred to as an electrode 110. The openings 108 within the cladding enable the electrode 110 (i.e., the exposed portion of the respective conductive structure) to contact tissue external to the cladding at or around the one or more openings 108, such as a blood vessel wall, or the like. The neural interface fiber 100 may have different dimensions and configurations depending upon the application. For example, the range of dimensions for the cross section of the neural interface fiber 100 may be: width X of the fiber: 50 μm-8 mm and thickness Z of the fiber: 25 μm-2 mm.

The plurality of conductive structures 104 extend substantially parallel to one another along a length of the neural interface fiber 100 and are embedded within cladding 102. The electrodes 110 are defined along an outer surface of neural interface fiber 100 and may be arranged in a specific non-limiting pattern as shown. As mentioned above, the electrodes 110 are exposed conductive structures 104 within respective openings 108 in the cladding 102 and are operable to deliver electrical impulses provided by an electrical signaling/recording device to stimulate tissues through a blood vessel wall, or other anatomical channels. To make the openings 108 in the cladding 102, the location of the openings 108 may be formed using laser etching, laser ablation techniques, or the like to expose the electrodes 110 at the desired locations.

The conductive structures 104 may include a plurality of conductive microwires. A conductive microwire of the plurality of conductive structures may be a carbon-based filament, platinum, tungsten, organic material including conducting polymers, doped polymers, carbon-polymer blends, platinum iridium (PtIr), combinations thereof, or the like. Examples of conducting polymers include poly (3,4-ethylenedioxythiophene) polystyrene sulfonate (PEDOT: PSS), and the like. The conductive structures 104 are selected for their low electrochemical impedance, a high charge storage capacity, and/or a high charge injection capacity to enable a signal applied to a proximal end of a conductive structure to be conducted to a distal end of the conductive structure without excessive loss of signal strength. In an example, the carbon-based filament or other conductive material forming the conductive microwire are configured to enable signals generated by the electrical signaling/recording device to provide a stimulation signal to the tissue without needing additional amplification or repeating along the length of the conductive structure 104, and return feedback signals generated in response to the stimulation signal or from tissues of interest. The respective conductive structures 104 are operable to carry signals generated by the electrical signaling/recording device an entire length of the respective conductive structure from the electrical signaling/recording device to the end cap (both shown in a later example).

A purpose of the conductive structures 104 is to enable signals generated by an electrical signaling/recording device (not shown in this example) to provide a stimulation signal via the electrodes 110 that contact tissue (not shown in this example) in an area of interest and enable recording of feedback signals generated at the area of interest.

In an embodiment, an additional element 112 may also be encapsulated in the cladding 102 extends along a length of the cladding and is positioned adjacent to the conductive structures 104. The additional element 112 may have a number of different uses and/or be filled with various materials. For example, the additional element 112 may be an opening, pathway, tunnel, microfluidic channel, or the like that permits the following: a chemical, medicament, mineral, or the like to be delivered to a target site location, and/or insertion of a rigid element for strengthening and adding rigidity to the cladding 102, a controllable element to facilitate positioning of the neural interface fiber, electrodes, or the like, another type of sensor (e.g., a camera, ultrasound device, radio frequency device, audio device, or the like), a lighting device, a radiopaque material, a heating element, a cooling element, a mechanical element, or the like.

The additional element 112 may be, for example, a shape memory portion in the form of a wire that extends along a length of neural interface fiber 100. The shape memory portion may be a shape memory alloy or shape memory polymer. For example, a shape memory alloy may be embedded as the additional element 112 in the cladding 102 for the formation, for example, of a different structure along the length, or at an end of the neural interface fiber 100. Examples of shape memory alloys include Nitinol, a copper-aluminum-nickel shape memory alloy, or the like. Examples of one or more shape memory polymers to be embedded in the cladding for the formation of a helical structure may include polyurethane, polyesterester, polyesterurethane, polyester, or poly (¿-caprolactone)/poly (butylene terephthalate). A “shape memory polymer” may have shape memory characteristics by virtue of its composition, the manufacturing process for the formation of the same, the presence of one or more dopants therein.

A distal end 106 of the neural interface fiber 100 may include, after a drawing process, exposed ends of the conductive structure 104. In some examples, the distal end 106, which is the end of the neural interface fiber 100 typically inside the subject's body, is covered by an end cap (shown in a later example). However, it is envisioned that the distal end 106 may be used inside the body.

The additional element 112 may include radiopaque material. The radiopaque material may be formed, for example, from bismuth subcarbonate; barium sulfate; bismuth oxychloride; bismuth trioxide; tungsten, gold, tantalum; and/or tantalum oxide, or any of these materials compounded with a polymer. In addition, or alternatively, radiopaque markers that are small quantities of radiopaque material may be used. For example, as the exposed ends of the conductive structures 104 at the distal end 106 of the neural interface fiber 100 may need to be covered.

The distal end 106 of the neural interface fiber 100 is the end portion that is to be implanted into a subject's blood vessel for neuromodulation. It is envisioned termination points at the distal end 106 of the plurality of conductive structures 104 (i.e., the exposed end portion of conductive structures 104 shown at the front cross-section of the neural interface fiber 100) may be operable to output a stimulation signal and/or receive a feedback signal. The front cross-section with the exposed conductive structures 104 may be considered a stimulation surface that is operable to deliver the neuromodulation signals generated by the electrical signaling/recording device.

Similarly, termination points at a proximal end 114 of the plurality of conductive structures 104 and operable to couple to an electrical signaling/recording device. The termination points at the proximal end may also be formed in the cladding 102 and provide access to the plurality of conductive structures 104 at one or more locations along through the cladding 102 and along the length of the neural interface fiber 100. In an example, the termination points are operable to couple to an electrical signaling/recording device (shown in another example.

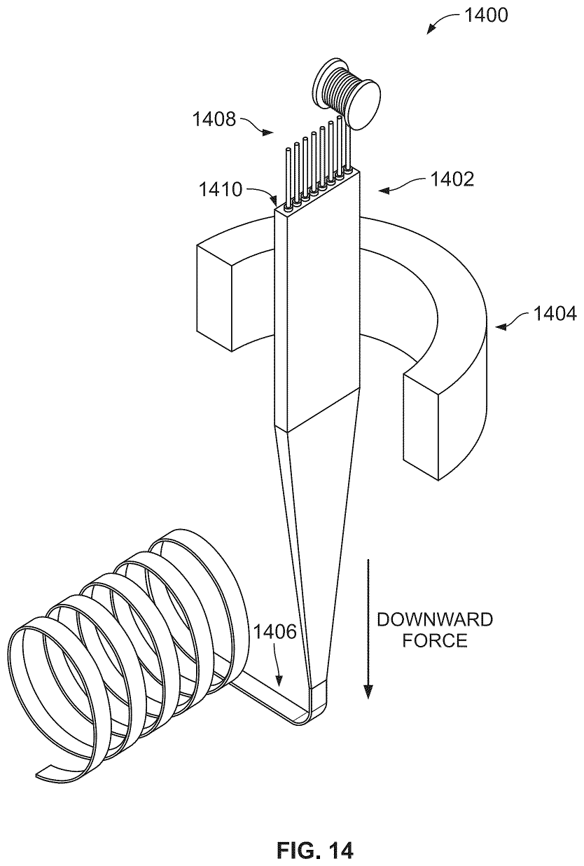

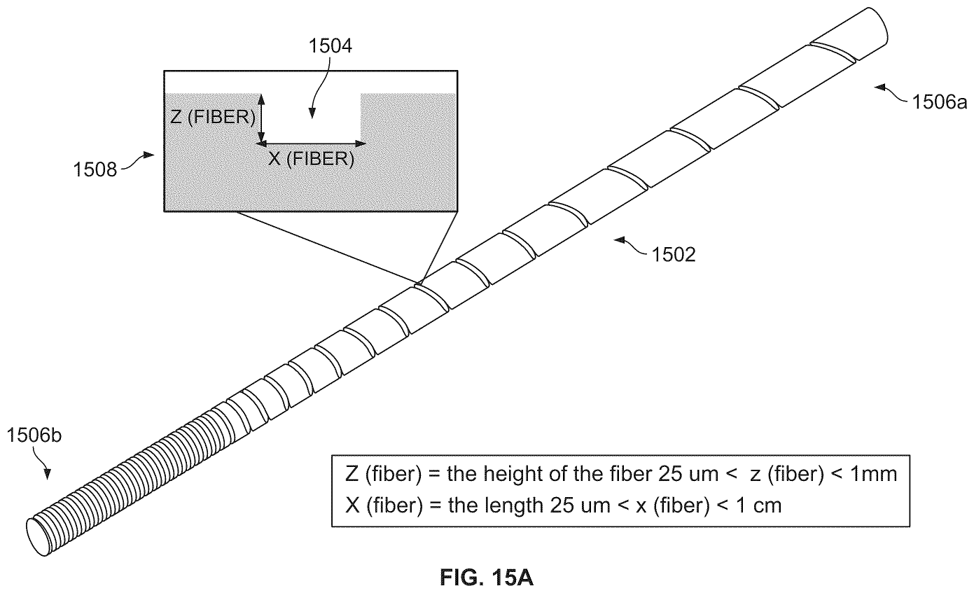

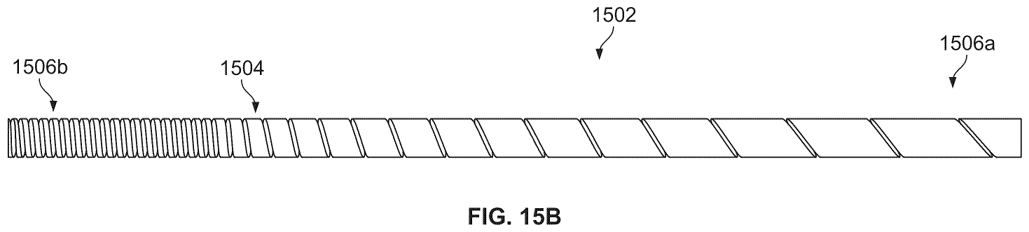

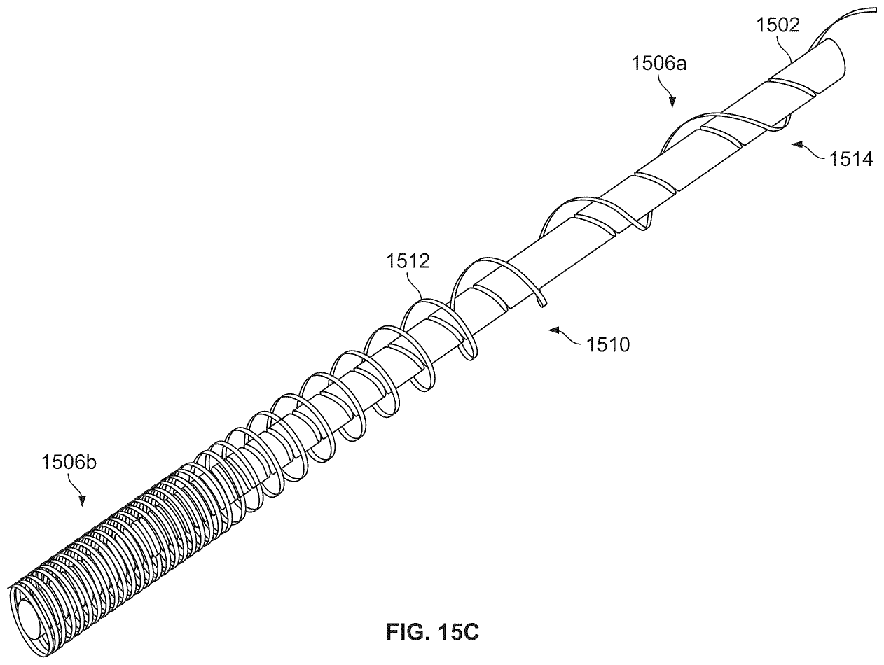

According to some embodiments, the provision of electrodes, including any additional processing thereof, such as the addition of layers, the provision of radio opaque markers and plugs may be performed after the drawing process of the neural interface fiber 100 (see e.g., FIG. 14-FIG. 15C) and before or after a shaping of the resulting structure into a helical form if a helical form is desired for the neural interface fiber.

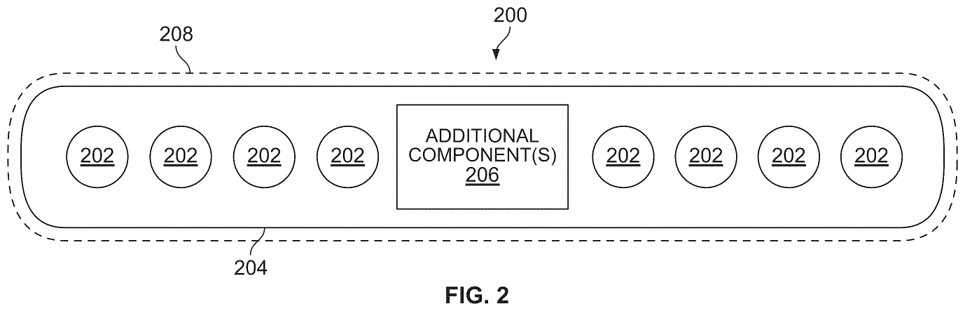

FIG. 2 illustrates an exemplary configuration of a neural interface fiber according to the disclosed subject matter.

The neural interface fiber 200 comprises conductive structures 202, cladding 204, and additional component(s) 206. For example, like neural interface fiber 100, the neural interface fiber 200 may have a range of dimensions for the cross section such as: a width X of 50 μm-8 mm and a thickness Z of 25 μm-2 mm.

The conductive structure 202 may be similar to the conductive structures 104 described above with reference to FIG. 1.

The shape of the cladding 204 may be a ribbon geometry with rounded corners to enhance blood compatibility and avoid thrombosis (i.e., blood clot) risk. In addition, the cladding 204 may be a dielectric insulation, such as a polymer. The cladding 204 may, for example, be a thermoplastic polymer having annealing properties that enable the cladding to be annealed into a number of different configurations and conformations.

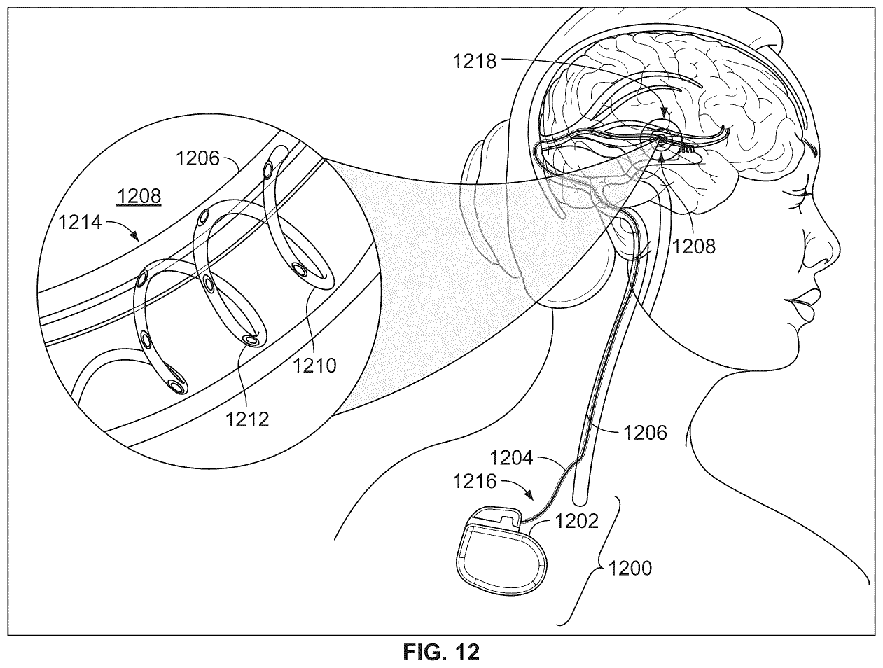

The additional component(s) may be functionally the same as the additional elements 112 of FIG. 12 except with a different arrangement. For example, the additional component(s) 206 may be continuous strands, compositions, or the like of material, such as a radiopaque material, which is visible to imaging devices, such as x-ray or the like, but do not form functional electrodes. For the example, the additional component(s) 206 may be formed from bismuth subcarbonate; barium sulfate; bismuth oxychloride; bismuth trioxide; tungsten, gold, tantalum; and/or tantalum oxide, or any of these materials compounded with a polymer. For example, a tungsten-polymer composition may be composed from tungsten powder such as 65 grams or the like that is combined in an elastomer or any thermoplastic polymer matrix, such as silicone rubber, or the like. In an example, an elastomer matrix is a polymer material that supports fibers, transfers stresses, and prevents crack propagation in composite materials. Similarly, any thermoplastic polymer that is suitable for incorporating tungsten or other radiopaque metal powders into may be used. In an example of making the additional component(s) 206, the load density of the tungsten powder with respect to the elastomer matrix is sufficiently high to enable radiopacity of the polymer composition and compatibility with manufacturing techniques, such as thermal fiber drawing, of the neural interface fiber.

In a further example, discrete radiopaque markers may be incorporated on the outside of the fiber as an additional layer 208. The neural interface fiber 200 can incorporate an additional layer 208 on the outer surface to enhance blood compatibility and reduce thrombosis (i.e., blood clot) risk. Alternatively, the surface of the neural interface fiber 200 may be chemically functionalized to achieve the same aim.

Advantageously, in another example, the capabilities of the neural interface fibers described herein may be enhanced by incorporating additional sensors (e.g., chemical sensors, imaging devices, or the like), actuation devices, such as a high-resistance wire to actuate shape-memory wire, and/or delivery devices (e.g., chemical delivery, electrical signals, or the like), such as microfluidic channel. Neural interface fiber 200 allows different types of delivery media to be provided deep into the tissue at precise targets. Examples of delivery media include chemicals, pharmacologic and/or genetic payloads. Incorporating the respective additional component(s) 206 to permit electrical stimulation, electrophysiology recording, light delivery for illumination, optical readout of fluorescent indicators or backscattered light, fast-scan cyclic voltammetry (FSCV) sensing of neurochemicals, or the like. These capabilities may be valuable in studies of neural circuits and for the development of clinically translatable neuromodulation approaches for the treatment of brain disorders. Having multiple capabilities and functions integrated within a single neural interface fiber is advantageous over a single-function neural interface fiber as it increases compatibility with long-term behavioral studies.

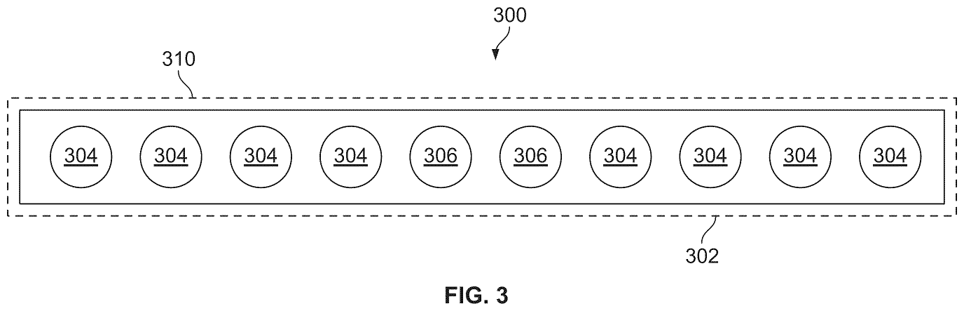

FIG. 3 illustrates another exemplary configuration of a neural interface fiber according to the disclosed subject matter.

The exemplary neural interface fiber 300 includes similar components as the neural interface fiber 200, such as cladding 302, conductive structures 304 and radiopaque material 306. Therefore, the discussion regarding FIG. 2 of the respective components applies to those that appear in FIG. 3. Differences between the exemplary neural interface fiber 200 of FIG. 2 and that of FIG. 3 are the shape of the cladding and the number of channels in the cladding and the shape of the channel for the radiopaque material. In an example, the neural interface fiber 300 may have a range of dimensions for the cross section, such as: a width X of 50 μm-8 mm and a thickness Z of 25 μm-2 mm, which are similar to those of neural interface fiber 100 and 200.

For example, the conductive structures 304 may be substantially similar to the conductive structures 202 of FIG. 2. However, the radiopaque material 306 is configured as at least two separate channels and that are configured in a cylindrical shape as opposed to the rectangular-shaped volume of additional component(s) 206 in FIG. 2.

The cladding 302 may be, for example, a thermoplastic polymer having annealing properties that enable the cladding to be annealed into a number of different configurations and conformations.

When implanted, because the squared edges of the cladding 302 may not be as blood compatible as the rounded edges of cladding 204 shown in FIG. 2, the neural interface fiber 300 may benefit from having an additional layer 310 that is either chemically treated to be blood compatible or shaped to be thrombosis resistant.

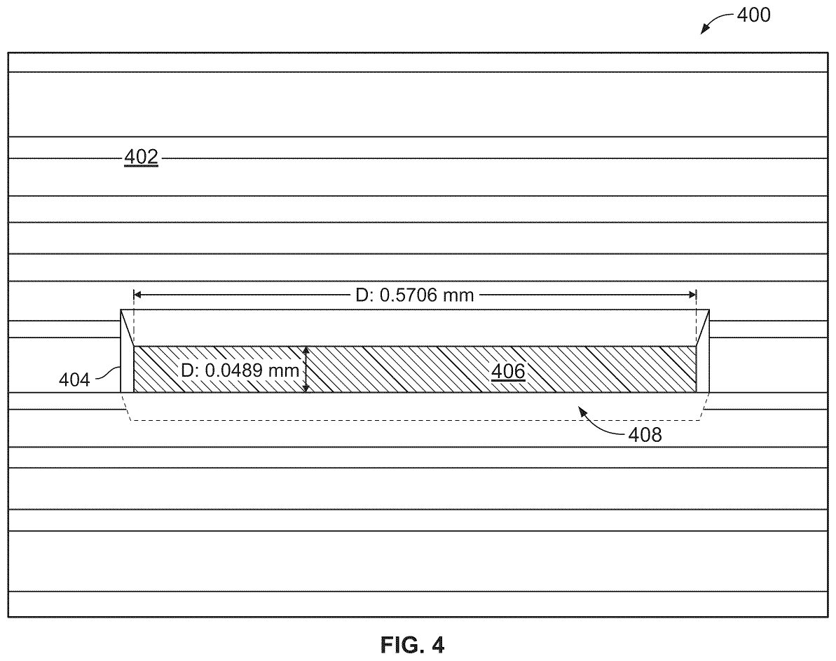

FIG. 4 illustrates a magnified view of an exemplary opening in cladding of a neural interface fiber to expose a portion of the conductive structure within in accordance with the disclosed subject matter.

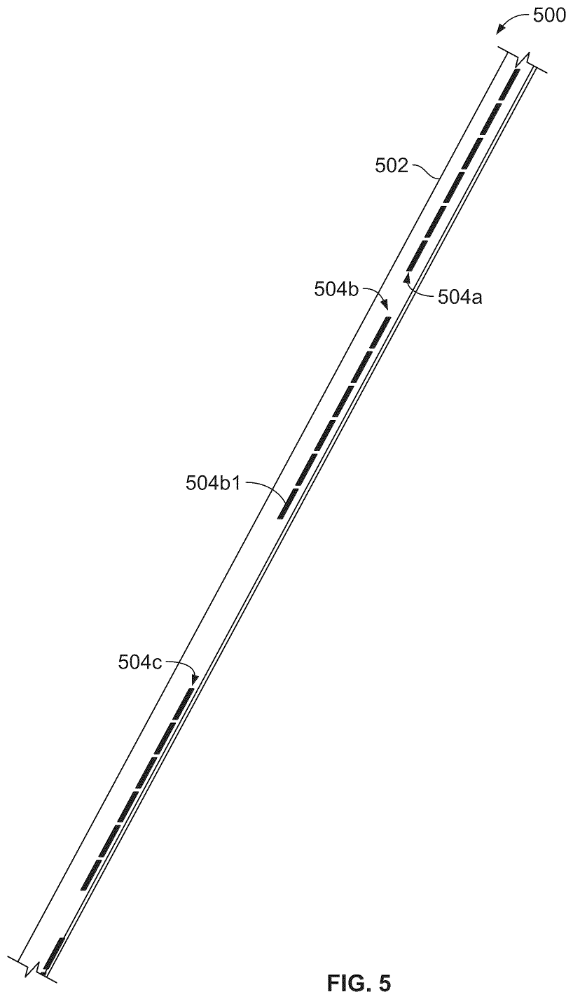

In this magnified view of an exemplary neural interface fiber 400, details of the cladding 402, an opening 404 in the cladding 402, conductive structure 406, and an electrode 408 are shown. The surface of the cladding 402 is shown to be substantially linear due to being drawn during manufacturing, which is discussed with reference to another example. The opening 404 extends from an exterior surface of the cladding 402 inward to an exposed surface of an electrically conductive structure embedded in the cladding 402, which is referred to as the electrode 408. The electrode 408 may be considered a single strip electrode, which is a single rectangular area in the opening 404 of the cladding 402. This is in contrast to a segmented electrode, shown in a later example, such as FIG. 5, in which each electrode is formed from multiple rectangular area in the opening of the cladding above the same filament within.

In an exemplary process to form electrodes, the openings 404 may be made by removing corresponding portions of the cladding 402 of the neural interface fiber 400 to expose the underlying electrically conductive structures and provide an electrode 408 by way of one or more of the following methods: laser etching or ablation; lithography, through the use of a mask, a photoresist, development of the photoresist and etching; focusing light beams onto the corresponding portions of the cladding in order to ablate the material thereof; mechanically removing the corresponding portions of the cladding, for example by drilling; and/or placing a mask onto the cladding to expose the corresponding portions, dipping the thus masked cladding into a chemical bath to dissolve/etch away the corresponding portions of the cladding, or chemical deposition, or the like.

In a detailed example, the electrode 408 is an outer facing surface of a conductive structure, such as a conductive microwire or the like, which is exposed to an environment of the neural interface fiber 400 via one or more of the openings 404 in the cladding 402. Different openings 404 may expose electrodes from different conductive structures beneath the cladding 402 of the neural interface fiber 400. For example, the electrode 408 may be exposed to air when the neural interface fiber 400 is not in use, or to the tissue of a subject when the neural interface fiber 400 is in use.

The thickness of the cladding 402 and the depth of the opening 404 are configured to enable the electrode 408 to contact tissue at or around the opening 404 and deliver any electrical signal by a electrical signaling/recording device (shown in another example) to or record any electrical activity from around the tissue by the electrical signaling/recording device.

The diameter of the microwire according to some embodiments, which may define a width dimension of an electrode formed by exposing a surface thereof, may be between about 10 microns and about 500 microns. For example, as shown in FIG. 4, the conductive structure 406 may have a diameter of approximately 0.05 mm (or 50 microns). The length of an electrode may be nearly any length depending upon the application for the neural interface fiber 400 according to some embodiments. For example, the length of the electrode 408 may be between about 10 microns and about 1 cm, and possibly between about 10 microns to about 2 cm. In the example of FIG. 4, the electrode 408 is approximately 570 microns, or 0.57 millimeters.

In one or more examples, a neural interface fiber 400 may have at least 1 electrode to about 64 electrodes at a surface of the neural interface fiber 400. When multiple electrodes are provided in a neural interface fiber, according to some embodiments, they may be distributed along an outer surface of the neural interface fiber in a manner consistent with intended neuromodulation uses of the neural interface fiber. For example, where the neural interface fiber has a helical structure (described in later examples), the electrodes may be disposed to face in different radial directions of a helical loop of the helical structure.

Additionally, when multiple electrodes are, for example, in a neural interface fiber, such as neural interface fiber 400, the multiple electrodes 408 may have different shapes and sizes with respect to one another. For example, a first portion of a length of a helical structure may have multiple similarly shaped electrodes, while a second portion of the length of the helical structure may have longer, shaped electrodes. Note that the shape of the electrodes 408 may vary with the shape of the openings 404 in the cladding 402. For example, the openings 404 and the respective electrodes 408 may be round, oval, rectangular, and have additional features such as rounded corners (e.g., half-circle rounds or the like) to mitigate crack propagation from the corners of the electrode opening or other stress-reducing shape features. The size of the electrodes and corresponding openings may be determined based on signal frequencies, signal strength, a combination of other signal characteristics, or the like.

It is also envisioned that some embodiments of a neural interface fiber include modifying a surface of an electrode of a neural interface fiber, such as electrode 408, to decrease the impedance of the conductive structure and increase the charge injection capacity (CIC) thereof, such as by way of one or more of the following: chemical etching to increase surface roughness at a micron scale, or addition of a hydrogel to the electrode surface, such as polyethylene glycol (PEG).

According to some embodiments, the electrodes 408 may be embedded in a barrier layer of the cladding 402 (not shown in this example) around them to improve their isolation with respect to one another. For example, when the conductive structures include carbon nanotubes, each conductive structure along the length of the neural interface fiber may have a layer including an electrically insulating material, such as polyimide, polycarbonate, or polyetherimide, around them.

Carbon nanotube (CNT) conductive structures that form electrodes may be used according to some embodiments to enable conventional electrophysiological recordings, localized electrical stimulation, and radiofrequency (RF) ablation, which demand a lower impedance profile than more conventional conductive structures (e.g., tungsten, carbon, or conductive polymer material). Carbon-based fibers that include carbon nanotubes used for electrodes, the low impedance and fast electron transfer kinetics of the CNTs allow efficient electrode neural stimulation and signal recording.

FIG. 5 illustrates a portion of an exemplary neural interface fiber with a pattern of electrodes according to the disclosed subject matter.

In this example, the portion of the neural interface fiber 500 includes cladding 502, electrodes 504a, electrodes 504b, and electrodes 504c. In this example, the cladding 502 may have openings that expose the underlying conductive structures to provide the respective electrodes 504a, 504b, and 504c. Each of the respective electrodes 504a, 504b, and 504c may include a number of individual electrodes, such as the 6 shown in this example, but may include more, such as 7, 10, 12, or the like. For example, each of the respective electrodes 504a, 504b, and 504c may be composed of a number of individual openings (e.g. 404 of FIG. 4) in the cladding 502. In a further example, the respective electrodes 504a, 504b, and 504c may be evenly distributed about the perimeter of a helical loop of the helical structure of a neural interface fiber.

The electrodes 504a, 504b, and 504c are examples of segmented electrodes. As shown in the exemplary figure, each electrode is formed with six electrode segments. For example, electrode 504b has six electrode segments, one of which is electrode segment 504b1. The segmented electrodes provide greater control of the neural interface fiber 500 and provide greater surface area for emitting stimulation signals and also receiving feedback signals for recording by an electrical signaling/recording device.

Similar to neural interface fiber 100 shown in FIG. 1, neural interface fiber 500 may have several conductive structures beneath cladding 502. In this example, the respective conductive structures are arranged parallel to one another. The first set of electrodes 504a are closer to a center of the neural interface fiber 100, and a bit further from center and closer to an edge of the neural interface fiber 100 is the second set of electrodes 504b, while the third set of electrodes 504c in substantially on the edge of the neural interface fiber 100. This configuration of electrodes provides for signal delivery and/or recording coverage over a distance and in different areas in which the neural interface fiber 100 contacts within a blood vessel.

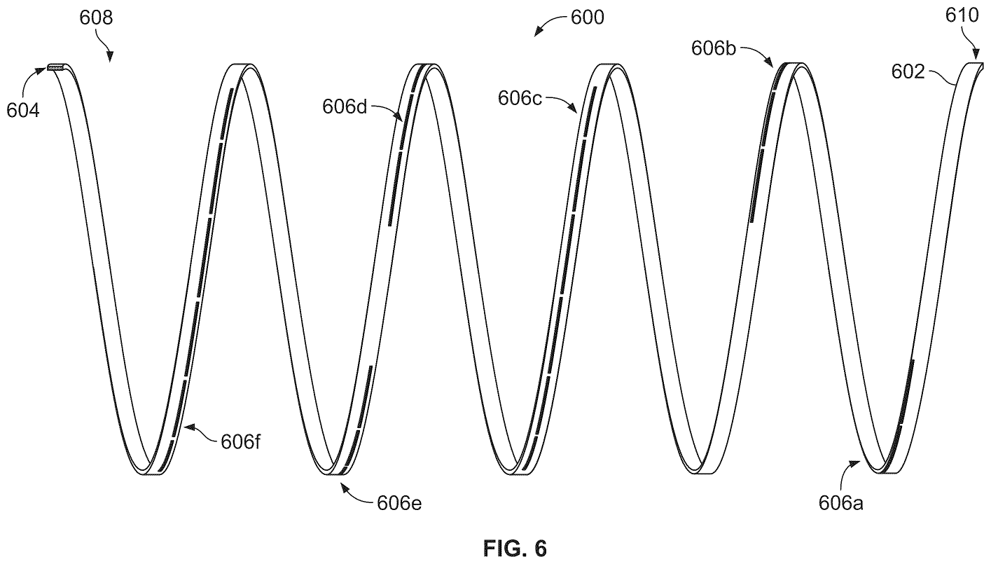

FIG. 6 illustrates a portion of an exemplary helical structure of the exemplary neural interface fiber of FIG. 5.

The exemplary helical structure 600 may include a cladding 602, conductive structures 604, and a number of electrodes 606a, 606b, 606c, 606d, 606e, and 606f.

As discussed with reference to earlier examples, openings may be made in portions of the cladding 602 exposing respective conductive structures 604 to provide the number of electrodes 606a, 606b, 606c, 606d, 606e, and 606f. The helical structure 600 is configured to expand to fill a space, such as a blood vessel. The helical structure 600 may have a proximal end 610 and distal end 608. The distal end 608 is the end portion that is to be implanted into a subject's blood vessel for neuromodulation. The proximal end 610 is closest to an electrical signaling/recording device and may have conductive structures 604 that extend out from the cladding 602 for coupling to the electrical signaling/recording device (not shown in this example). Alternatively, the conductive structures 604 may be accessible for coupling to the via openings in the cladding 602 that are adapted for connection to terminals of the electrical signaling/recording device.

Some embodiments include a plug material that is attached to, or formed on, exposed ends of the conductive structures 604 at the distal end 608 surface of the helical structure 600 to electrically insulate the ends of the conductive structures 604. For example, such plug material may form endcaps on the exposed ends of the conductive structures 604.

The electrodes 606a, 606b, 606c, 606d, 606e, and 606f may be used to provide the neuromodulation treatments to the tissue areas of the subject. The electrodes are shown in a strip configuration (i.e., single, elongated, rectangular areas in the openings of the cladding 602). In some examples, the electrodes are primarily distributed at a lower edge of the helical structure 600 of the neural interface fiber. A benefit of the strip configuration for an electrode is that it facilitates an increase in the available electrode surface area for tissue stimulation, which can be advantageous depending upon neuromodulation treatment application needs.

For example, where a neural interface fiber has a helical structure 600, the electrodes of the helical structure 600 may be disposed to face in different radial directions of a helical loop of the helical structure 600. In FIG. 6, the electrodes 606a, 606b and 606e, for example, may also be present on a back surface (i.e., the surface of the helical structure 600 that is not visible in the figure).

The spiral construction of the helical structure 600, and other helical structures described herein, enables electrodes thereon to have their exposed surfaces-surfaces that are facing an outer surface of the neural interface fiber and that are adapted to be in contact with vessel walls of a subject after implantation to better contact those vessel walls after implantation. In addition, a helical structure according to some embodiments advantageously holds the blood vessel open after implantation and serves to anchor the neural interface fiber in the blood vessel for better electrode contact and therefore better electrical stimulation.

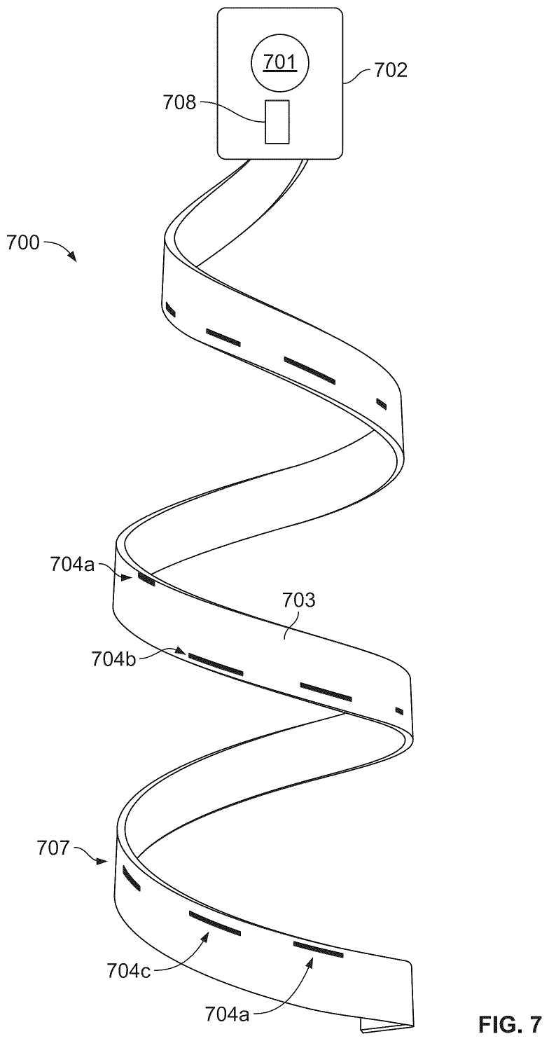

FIG. 7 illustrates a portion of an exemplary neural interface fiber according to the disclosed subject matter.

The exemplary neural interface fiber 700 includes an end cap 702 containing a guide wire hole 701, a cladding 703, multiple electrodes 704a, 704b and 704c, and helical loops 707. A plurality of the helical loops 707 may form a helical structure that is a portion of the neural interface fiber 700. For example, the helical structure may originate at a location close to or at the end cap 702 and extend a predetermined length from the end cap 702.

As shown and described with reference to earlier examples, a neural interface fiber 700 may have a helical structure. In this example, the neural interface fiber 700 has a rectangular cross section in a plane perpendicular to a length thereof (as will be explained in further detail in relation to FIG. 5 below), which imparts a ribbon configuration to the neural interface fiber 700.

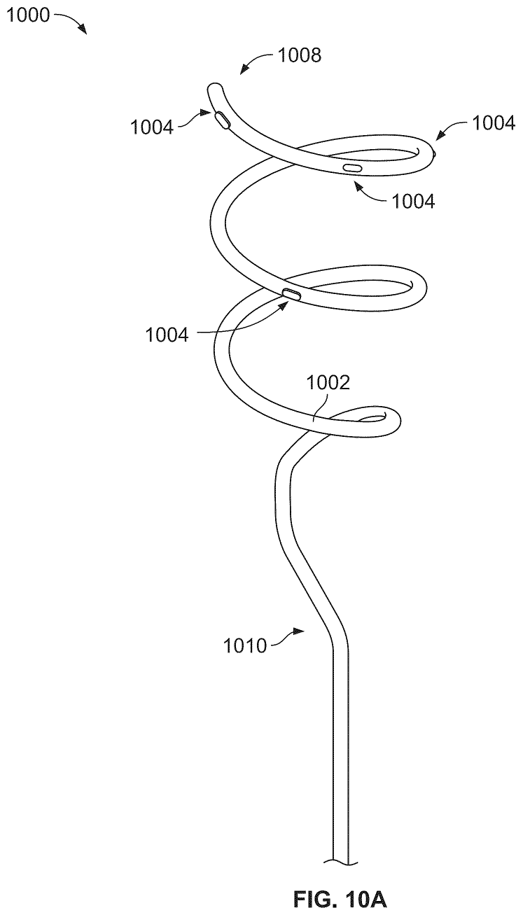

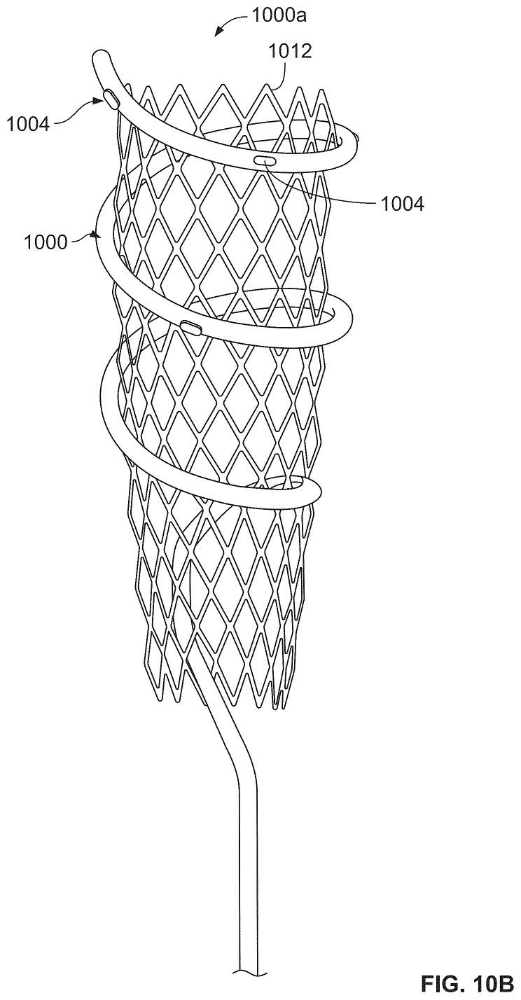

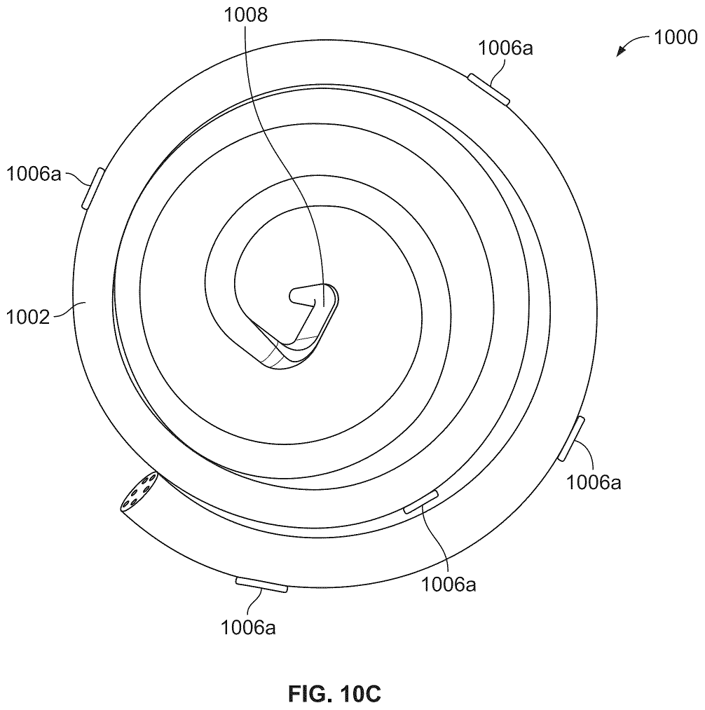

The neural interface fiber 700 has helical loops 707. The helical loops 707 may, according to one embodiment, be circular, as depicted in a perspective bottom plan view of FIG. 10C to be described in further detail below.

The cladding 703 of the neural interface fiber 700 is substantially similar to the cladding described with reference to the examples of FIGS. 1-6 above. The cladding 703, which is a dielectric insulation material, for example, is adapted to substantially encompass a plurality of conductive structures (as shown in the example of FIG. 1). The cladding may be a dielectric insulation. The cladding 703 includes one or more openings exposing a portion of a respective conductive structure within the cladding 703.

In this example, the electrodes 704a, 704b, and 704c may be different exposed portions of a respective conductive structure (shown in other examples). For example, the respective electrodes 704a, 704b and 704c are accessible through the openings (not shown in detail in this example) in the cladding 703. The exposed electrodes 704a, 704b, and 704c are operable to output signals to and receive signals from tissue that contacts the respective electrodes 704a, 704b, and 704c.

The respective electrodes 704a-c are arranged on the outer surface of the cladding 703. In the shown embodiment, the electrodes 704a-c are disposed at differing locations on the cladding 703. The electrodes 704a-c are shown having a single, elongated strip configuration, however, the shape of the electrodes 704 are not so limited, and may be circular, square, ovular, or the like. Alternatively, the respective electrodes 704a, 704b, and 704c may be implemented as segmented electrodes, where each respective electrode is formed from multiple rectangular areas in the opening of the cladding above the same conductive structure within.

The end cap 702 may be coupled to the end of the cladding 703. In the example, the end cap 702 may be fastened or affixed to a distal end of the cladding 703 and be configured to electrically insulate the plurality of conductive structures (not shown) within the cladding 703. The end cap 702 may include guide wire hole 701 and radiopaque material 708. In an example, a guidewire (not shown) may be positioned in the guide wire hole 701 to enable placement of the helical loops 707 near tissue for neuromodulation.

While the radiopaque material 708 is shown in FIG. 7 as being located in the end cap 702, the radiopaque material 708 may be present within a portion of the cladding 703 and may extend for part of, or substantially the entire length of the neural interface fiber 700. Alternatively, or in addition, radiopaque material 708 may be specifically positioned at locations along the neural interface fiber 700 as radiopaque markers as reference points for clinicians conducting the neuromodulation therapy. As described herein, radiopaque materials may be used in the end cap 702, along the entire length of neural interface fiber 700, as marker(s) at different points along the neural interface fiber 700, or a combination thereof.

Radiopaque material 708 may be located on the end cap 702 to provide a clearly visible tip marker when imaging is used, such as fluoroscopy or the like. The radiopaque material 708 may be incorporated into the end cap 702 and may include: bismuth subcarbonate; barium sulfate; bismuth oxychloride; bismuth trioxide; tungsten, gold, tantalum; and/or tantalum oxide, or any of these materials compounded with a polymer. In some examples, a radio marker may include X-ray opaque materials. For example, the opaque X-ray material may be mixed while in powder form with a polymer material, and the polymer material used to form the end caps end cap 702 on neural interface fiber 700 to provide both end cap 702 and radio markers (not shown in this example). In another example, the radiopaque markers may be formulated in paint form, which may be painted on the end cap 702 already provided on a neural interface fiber 700.

In a specific example, the neural interface fiber 700 has a helical structure at an end that has a number of helical loops 707. The neural interface fiber 700 includes a cladding 703 similar to that described and shown in relation to other figures. Electrically conductive structures, such as 104 of FIG. 1, may be, for example, in the form of a plurality of parallel microwires that extend along the length of the neural interface fiber 700 and are embedded within cladding 703. Electrodes 110 may be defined along an outer surface of neural interface fiber 100 in a specific non-limiting pattern as shown. Electrodes 110 are within recesses/openings 108 in the cladding 102, which may be provided by way of any well-known method, such as laser etching, or through other methods as will be explained herein. In an example, the neural interface fiber 700 may include an additional element, such as the additional element 112 of neural interface fiber 100 in FIG. 1. In this example, the additional element may be a shape memory portion, which may be in the form of a wire that extends along a length of neural interface fiber 700. The shape memory portion (i.e., additional element 112) may include any one or more of the shape memory materials described above.

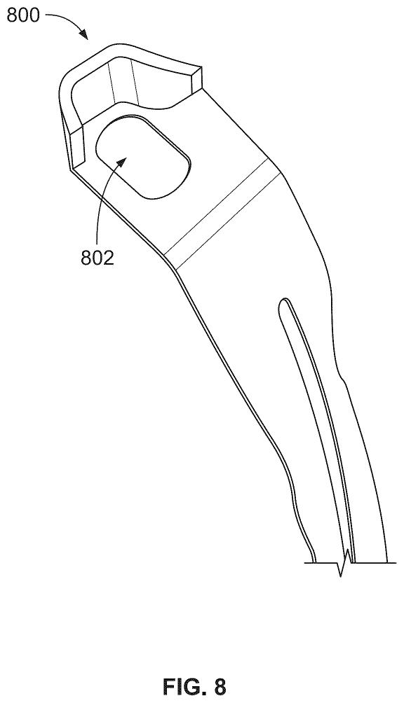

FIG. 8 illustrates an exemplary end cap with an exemplary delivery feature according to the disclosed subject matter.

The end cap 800 may be added onto the neural interface fiber to serve several purposes. For example, it may electrically insulate any exposed conductive structures, such as conductive microwires, at the end of the neural interface fiber thereby only allowing any of the conductive structures to only be exposed at the desired electrode locations along the surface of the fiber. As shown in FIG. 7, radiopaque materials, such as 708, may be incorporated into the end cap 800 to create a clear tip indicator (under imaging) to enhance clinical usability. A plug material may be placed on the exposed ends of the conductive structures 104 at the distal end 106 to electrically insulate the exposed ends of the conductive structures 104. The plug material may, for example, include a material, such as a polymer, which is similar to the dielectric insulation of the cladding 102.

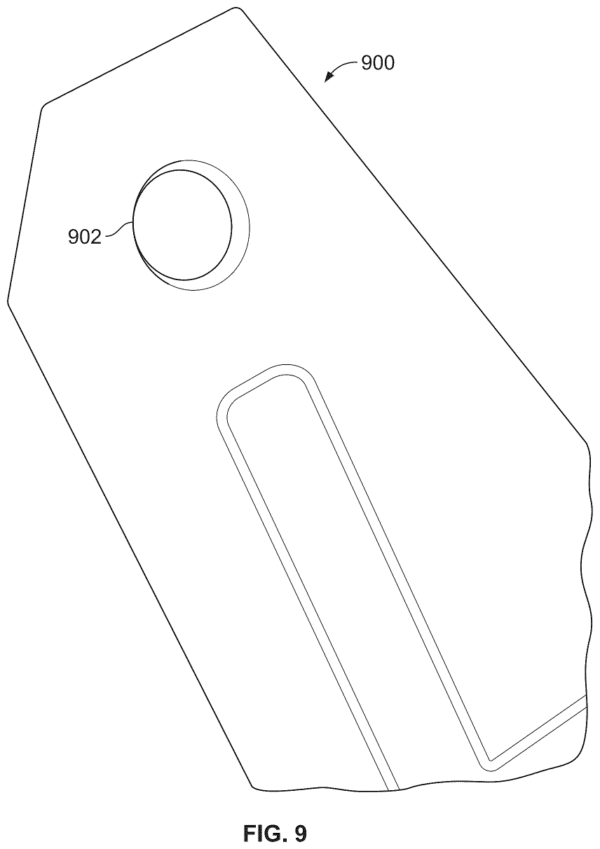

FIG. 9 illustrates a detailed view of an exemplary end cap with an exemplary delivery feature according to the disclosed subject matter.

In the detailed view, the end cap 900 may be configured with different features to facilitate endovascular delivery. For example, the end cap 900 may be configured with a small hole 902 or loop which can be hooked onto a guidewire or delivery mechanism (not shown) to push the neural interface fiber into the desired vessel location and then released. In an example end-cap manufacturing process, the end cap 900 may be formed, for example, by laminating a medical grade epoxy between thin films of thermoplastic polymer. The thermoplastic polymer may be the same polymer used for making the cladding of the exemplary neural interface fiber (shown in other examples).

FIG. 10A illustrates a portion of another exemplary helical structure of an exemplary neural interface fiber according to the disclosed subject matter.

The helical structure 1000 may include a cladding 1002, an electrode 1004, a distal end 1008, and a proximal end 1010.

The cladding 1002 is an outer surface of the helical structure 1000 and may have electrodes 1004 on the outer surface of the cladding. In the shown embodiment, there are four electrodes 1006a shown, which are facing in different radial directions of the shown helical structure. The helical structure 1000 includes a proximal end 1010, which is the end portion of the helical structure 1000 closest to a connection with a controller including processing circuitry to activate the electrodes 1004, and a distal end 1008, which is the end portion that is to be implanted into a subject's blood vessel and/or tissue for neuromodulation. The helical structure 1000 also includes a helical structure at the distal end 1008, and a non-helical portion leading to its proximal end 1010.

In this example, the electrodes 1004 are shown as exposed surfaces having an oval configuration. Of course, the electrodes 1004 may have other configurations, such as rectangular, square, circular, or the like.

In an example, the electrodes 1004 may be in the cladding 1002 at outer surfaces of a helical structure 1000 of the portion of the neural interface fiber. In the example of FIG. 10A, the electrodes 1004 may have a strip configuration, and are primarily distributed at a lower edge of the helical structure of the neural interface fiber. The strip configuration facilitates an increase in the available electrode area surface for tissue stimulation, which can be advantageous based on application needs. For example, the positioning of the electrodes 1004 on the perimeter of a helical loop facing in opposite directions may provide granularity in terms of control over available directions for neurostimulation.

Other configurations of a helical structure are also envisioned. FIG. 10B illustrates an exemplary modified helical structure in FIG. 10A according to the disclosed subject matter.

In the modified portion of the modified helical structure 1000a, the helical structure 1000 (either along an entire length thereof, or along only a portion thereof, such as a distal portion thereof) may, during its deployment in tissue, be expanded by an elongated expansion structure, such as expansion structure 1012. The expansion structure 1012 according to some embodiments may correspond to a flexible body, such as, a flexible mesh body akin to a vascular stent, or such as, by way of example only, a balloon body akin to an angioplasty balloon. In an operational example, the expansion structure 1012 may be deployed within the helical structure 1000 with the electrodes 1004 positioned on an outer portion of the helical structure 1000 facing outward from the expansion structure 1012. When the expansion structure 1012 is deployed by expanding, the electrodes 1004 may be positioned or implanted at a target site within a blood vessel. When expanded the expansion structure 1012 is anchored at the target site, thus establishing among other things better electrodes 1004 contact with tissue (not shown in this example) by pushing the electrodes 1004 against inner blood vessel walls.

As shown, the expansion structure 1012 may include a mesh configuration. In an example, at least some portions of the inner surfaces of the cladding (not shown in this example) may include grooves. The cladding grooves may correspond to the shape of the mesh configuration of the expansion structure 1012, when expanded. This would facilitate securing the expansion structure 1012 in the cladding of the helical structure 1000 of the neural interface fiber.

An alternative to the mesh configuration of the expansion structure 1012 may be a balloon device that may be similarly configured and serve the same purpose.

In some examples, the expansion structure 1012 and the helical structure 1000 are implanted together at the same time. For example, the expansion structure 1012 is prepositioned within the helical structure 1000 before implantation and guided to the target site location in a vessel for deployment. In other examples, the expansion structure 1012 may not be positioned contemporaneously with the helical structure 1000. In this alternate operational example, the expansion structure 1012 may be advanced to the target site location separately from the helical structure 1000 and the neural interface fiber. Either the expansion structure 1012 may be positioned first or alternatively, the helical structure 1000 may be positioned at the target site location without yet engaging the blood vessel wall and the expansion structure 1012 may subsequently be positioned within the helical structure 1000 and expanded to affix the electrodes 1004 to the target site location on the blood vessel wall.

FIG. 10C illustrates a bottom plan view of an exemplary portion of the exemplary helical structure of the example of FIG. 10A. The bottom plan view is from a proximal portion of the neural interface fiber toward its distal end 1008.

The electrodes 1006a may be at outer surfaces of cladding 1002 of the helical structure 1000 of a neural interface fiber. Electrodes 1006a are shown facing different radial directions from a central axis of the helical structure 1000, which may enable each electrode of the number of electrodes 1006a to individually stimulate tissue in different directions based on a control algorithm (e.g., a stimulation control algorithm as described in a later example) executed by the electrical signaling/recording device. For example, direction of the stimulation may be based on selection of an appropriate pair of electrodes within the helical structure 1000.

Electrodes 1006a are shown as protruding from the outer surface of the cladding. The protrusion of the electrodes 1006a may be built by providing additional electrically conductive layers over the material of the electrically conductive structures (e.g., conductive microwires or the like) within openings, such as 108 or the like, in the cladding 1002. The provision of such electrically conductive layers may be through any known method, such as by way of electroplating or the like.

A mean or median helix diameter of the exemplary helical structure 1000 portion of the neural interface fiber according to some examples may be based on a diameter of a blood vessel to be accessed. For example, the mean or median helix diameter of a neural interface fiber may be between about 250 microns to about 3 centimeters, between about 500 microns to about 2 centimeters, or the like. This mean or median helix diameter may correspond to a vein or artery in the neck, leg, abdomen, brain, or heart of a neuromodulation subject.

The electrodes 1006a may be located at outer surfaces of the cladding 1002. Alternatively, as shown in the example of FIG. 1 and FIG. 4, the electrodes are within openings, such as openings 108 and 404, defined in the cladding of the neural interface fiber, the openings expose the respective conductive structures at each respective opening.

As shown, the electrodes 1006a may have outer surfaces that protrude away from an outer surface of the cladding 1002. In this example, the electrodes are not only exposed via openings in the cladding 1002 but may have outer surfaces that protrude away from an outer surface of the cladding 1002. In the example, the electrodes 1006a may be provided by depositing additional layers or films of electrically conductive material on exposed surfaces of the respective conductive structures (e.g., microwires) embedded within the cladding so the electrodes are at or above the surface of the cladding 1002. Deposition of such layers may be done by way of, for example, electroplating, or the like. Possible materials to electroplate onto the electrode surface to decrease the electrode impedance and improve the charge injection capacity (CIC) may include: gold, platinum, platinum black, carbon black, Poly (3,4-ethylenedioxythiophene) polystyrene sulfonate (PEDOT: PSS), Iridium oxide, single walled or multi walled carbon nanotubes; Polyethylene glycol (PEG), and/or the like.

Depositing processes, such as electroplating, chemical vapor deposition, lithographic approach, e-beam, deposition, sputtering, laser-induced carbonization of the cladding, or the like, may be used to microfabricate the electrodes 1006a. For example, when using electrodeposition of material to form the electrode, the conductive microwire within the neural interface fiber may be conductive material, such as stainless steel, gold, any metal, alloy, or non-metal conducting microwire. Additionally, during the depositing process, mechanical compliance and cladding integrity may be maintained by limiting thickness and/or applying barrier or adhesion-promoting layers between the microwire and the electroplated material to prevent delamination.

According to some embodiments, the electrodes on a neural interface fiber with a helical structure may each have a length that, for example, spans about ¼ of a helical loop, ½ of a helical loop, ¾ of a helical loop, or all the helical loops (for spherical stimulation). For example, an electrode surface may span, according to an embodiment, every half turn of a helical structure of a neural interface fiber. For example, eight electrodes may be arranged to take up about four loops or turns of the helical structure 1000. In other examples, the number of electrodes and loops or turns of helical structure may vary. For example, a four-turn helical structure may include 1-4 electrodes of a same given length, or 1-4 of various lengths. Alternatively, the number of turns of the helical structure may vary from 2-20 turns depending upon the neuromodulation treatment and/or the target site location.

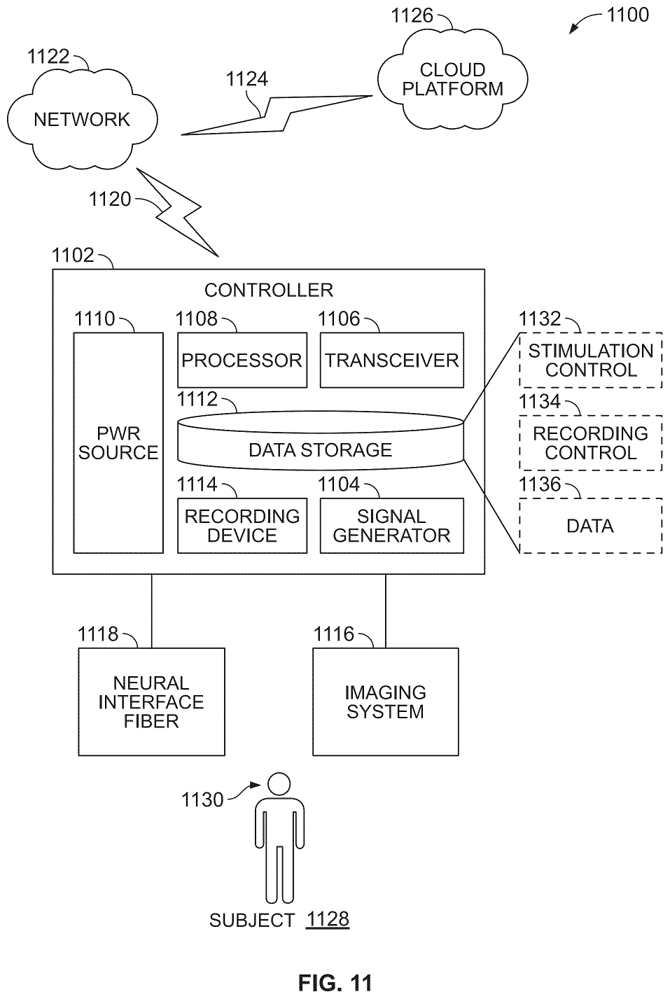

FIG. 11 illustrates a block diagram of a neuromodulation system utilizing the exemplary neural interface fibers of the disclosed subject matter.

The neuromodulation system 1100 may include a controller 1102, a signal generator 1104, a transceiver 1106, a processor 1108, a power source 1110, a data storage 1112, and a recording device 1114. The neuromodulation system 1100 includes inputs for a neural interface fiber 1118, and optionally, an imaging system 1116. The neural interface fiber 1118 may be configured as described with reference to earlier examples and operable to couple to signal generator 1104 and/or the recording device 1114.

The imaging system 1116 may be a fluoroscopic device, which is operable to be able to track radiopaque markers, such as those described in other examples, for example, in an end cap or an additional element or layer within or on the cladding of the neural interface fiber. The imaging system 1116 includes a display device that enables an operator of the neuromodulation system 1100 to track progress of the neural interface fiber 1118, the helical structure and/or end cap in particular, to the target site location for neuromodulation therapy (e.g., stimulation and recordation).

In more detail, the controller 1102 may include processing circuitry, such as processor 1108. The processor 1108 may be coupled to the data storage 1112, which may be a memory device operable to store programming code, such as a stimulation control algorithm 1132, and a recording control algorithm 1134. The data storage 1112 may also store data 1136. The programming code when executed by the processor 1108 is operable to cause the processor 1108 to perform functions as described herein and related to neuromodulation, such as causing signals to be generated, via the stimulation control algorithm 1132, by the signal generator 1104, and the recording of signals, via the recording control algorithm 1134, by the recording device 1114. The data 1136 may include a history of the signals generated for a specific neuromodulation treatment session and/or a record of signals recorded in response to or during the specific neuromodulation treatment session.

The subject 1128 may be a patient that is being treated using neuromodulation provided by neuromodulation system 1100.

For example, the controller 1102 may include processing circuitry, such as processor 1108, to activate the electrodes (shown in earlier examples) at a distal end of the neural interface fiber that is to be implanted into, or positioned in, a blood vessel of the subject 1128 for neuromodulation.

The transceiver 1106 is operable to establish and communicate with a network 1122 via communication link 1120. The transceiver 1106 may also be operable to communicate with a cloud platform 1126 via, for example, communication link 1124. The network 1122 may be any form of data network, such as a local area network, a wide area network, a campus network, a cellular network, the internet, or a combination thereof. The network 1122 may facilitate connection with a vendor or backend system, which may be hosted on a cloud platform 1126. The cloud platform 1126 may provide signal generation information (i.e., modulation patterns, voltage settings, or the like) and/or recording analysis and cloud storage for use by neuromodulation system 1100.

The instructions stored within the data storage 1112 may cause the processing circuitry, when in operation, to activate one or more of the electrodes in order to provide stimulation to specific locations of the target area, such as stimulation area 1130, of subject 1128. The instructions stored within the memory may further cause the processing circuitry to activate the electrodes to record, by the recording device 1114, electrophysiological signals generated by the subject 1128 at the target area of neural interface fiber 1118 implantation. The processing circuitry may store the recorded electrophysiological signals in the data storage 1112 of the controller 1102. In addition to storing instructions that enable controlling output of the different voltages output from the signal generator 1104 to the neural interface fiber 1118, the data storage 1112 may also store instructions that enable the processor 1108 to analyze and evaluate the recorded electrophysiological signals.

In the prior examples, the neural interface fiber 1118 is described as including conductive structures, such as conductive microwires, that extend along at least part of a length of the neural interface fiber 1118. Additionally, at parts of the neural interface fiber 1118 there are electrodes, as described herein. The neural interface fiber 1118 has a distal end and a proximal end. In those examples, the proximal end is the end portion of the neural interface fiber 1118 closest to the controller 1102. The proximal end of the neural interface fiber 1118 may have conductive structures that couple to the controller 1102 to be supplied with stimulation signals from the signal generator 1104 and to provide signals detected from the stimulated tissue to be recorded by the recording device 1114. Alternatively, or in addition, the neural interface fiber 1118 may have, for example, additional exposed regions (formed, for example, by laser ablation) in its cladding at which the controller 1102 is operable to be coupled to the neural interface fiber 1118 and access exposed conductive structures to apply a stimulation signal and/or receive feedback signals for recordation.

In an operational example, to create temporal interference, a controller 1102 may cause the signal generator 1104 to output signals to the electrodes of respective conductive structures of an implanted neural interface fiber independently at respective frequencies so that the frequencies constructively interfere in the target area. Alternatively, different voltages may be applied to different respective electrodes (of different respective conductive structures) within the same neural interface fiber in a bipolar manner to create a similar effect. For example, the controller 1102 may be operable to optimize stimulation signals at respective target site locations to obtain a desired interference pattern utilizing a pair of neural interface fibers 1118. For example, the controller 1102 optimizes stimulation signals applied by the signal generator 1104 from each fiber based on imaging data from the imaging system 1116, and knowledge of a distance between respective electrodes of the respective neural interface fibers of a pair of neural interface fibers 1118, and/or feedback from recorded interference data resulting from respective stimulation by the two neural interface fiber 1118. The recording device 1114 may be operable to record electrical feedback signals produced in response to the application of the stimulation signal applied to tissue at or around a target site location.

The temporal interference generated in areas, such as stimulation area 1130, using examples of the neural interface fibers 1118, as described herein, enables a higher spatial resolution at the target site location of the subject 1128 as compared with temporal interference administered from a scalp surface of the subject 1128, thereby allowing a better focusing of the target area, and allowing the focus of the interference field to be more readily steered.

In addition, the processor 1108 may also be operable to use the recorded electrophysiological data from the electrodes to determine a metric (such as signal strength, signal duration, frequency information, or the like) that is used to control the signals output by the signal generator 1104 (i.e., stimulation output) to the electrodes of the neural interface fiber 1118. The controller 1102 may independently address (i.e., cause the signal generator 1104 to output a stimulation signal) individual ones of the electrodes of the neural interface fiber 1118, for example, based on analysis and evaluation of the recorded electrophysiological data received via the electrodes.

According to some embodiments, each electrically conductive structure within the cladding (e.g., each microwire) may be independently activated by the controller 1102 via a stimulation control algorithm 1132, which enables targeted localized stimulation of a target site location (i.e., stimulation area 1130) via an electrode when the neural interface fiber 1118 is implanted at a target location site of the subject 1128. Independent activation of respective electrodes may mean applying a given voltage and using different activation parameters to allow individual tuning of stimulation signals output from each electrode.

Different types of power sources may be used to power the 1100. For example, the power source 1110 may be a battery, either rechargeable or changeable, a piezoelectric or other energy harvesting device, a solar device, or the like.

FIG. 12 is a graphic illustration of an exemplary implementation of the disclosed exemplary neural interface fibers.

A neuromodulation system 1200 may be implanted beneath the skin of a subject and may include a controller 1202 and a neural interface fiber 1204 as described in earlier examples. The implementation may take place in a blood vessel 1206 with a terminus of the helical structure 1210 of the neural interface fiber 1204 at a target site location 1208. The output from the electrodes 1212 may stimulate tissue in a stimulation area 1230 and the electrodes 1212 may also be operable to receive feedback signals.

A detailed view of the target site location 1208 shows the positioning of the helical structure 1210 within the blood vessel 1206.

The controller 1202 may be an implantable pulse generator (IPG), and at least part of the processing circuitry (described with reference to FIG. 11) may be part of the IPG. The controller 1202 may include one or more transceivers configured for either wired or wireless communication. In an example, the controller 1202 may be configured to be charged wirelessly.

The controller 1202 may include a signal generator, such as an implantable pulse generator (IPG). The controller 1202 may include one or more communication units, such as a transceiver 1106 of FIG. 11, which may be a wired or wireless device. The controller 1202 may further include a power source, such as a power source 1110 of FIG. 11, that may have one or more inputs for charging and may further be configured to be charged wirelessly.

The detailed view of the target site location 1208 shows an exemplary clinical environment implementation of a neuromodulation system 1200 is which a stimulation area 1218 is undergoing a clinical procedure in-vivo. In the detailed view, a distal end of the helical structure 1210 is inserted into a blood vessel 1206 of the stimulation area 1130, and directed to the target site location 1208 for stimulation or recording as the distal end 1214 is in the blood vessel 1206. As shown, the electrodes 1212 are positioned, for example, against a wall of the blood vessel 1206 and are operable to stimulate tissue in the target site location 1208. The stimulated tissue may be in the stimulation area The proximal end 1216 of the neural interface fiber 1204 is connected to controller 1202.

To implant the neural interface fiber 1204, according to some embodiments, a catheter (not shown) of a custom-designed catheter deployment system (e.g., a guidewire as described with reference to the examples of FIG. 7, FIG. 8 and FIG. 9) may be used to navigate to the target site location 1208 in the blood vessel 1206. Thereafter, the helical structure 1210 may be decoupled from the catheter when positioned in the blood vessel 1206.

While the foregoing is a specific example, other neuromodulation implementations and techniques are also considered and may be utilized.

A vestibular migraine is often diagnosed when vertigo occurs during a migraine headache, or shortly before or after one. It remains unclear precisely what causes a vestibular migraine, but one hypothesis is that neural connections between the sensory systems, which process head pain, and the vestibular systems, which establish a sense of spatial awareness and balance, could be communicating during a migraine attack thereby exacerbating or intensifying the migraine attack.

Treatment options for vestibular migraines are extremely limited, so current treatment is focused primarily on reducing the frequency of migraine attacks. Emerging research suggests that a new application of an existing treatment may hold some promise. The treatment, called noninvasive VNS (nVNS), involves placing a small handheld device over a subject's neck to deliver a short electrical pulse to activate their vagus nerve. The vagus nerve is a long nerve that descends from the subject's brainstem through their neck and regulates all organs in your their including your heart rate, gut motility, and mood. Additionally, electrical stimulation of the vagus nerve is used to treat both epilepsy and depression.

However, there are limits to how long and how often a subject may be able to keep the small handheld device in the area around the vagus nerve. Additionally, the placement of any handheld device may vary, and results may differ from application to application of the device.

Hence, since stimulation of the vagus nerve may treat not only migraines but also epilepsy, depression, sepsis and septic shock, rheumatoid arthritis, Crohn's disease, irritable bowel syndrome, postoperative atrial fibrillation (POAF), heart failure, and more. Many of these conditions are treated by tapping into the inflammatory reflex by which the vagus nerve modulates systemic inflammation in the body. For example, an implantable neuromodulation system may be preferred such as neuromodulation system 1200 of FIG. 12.

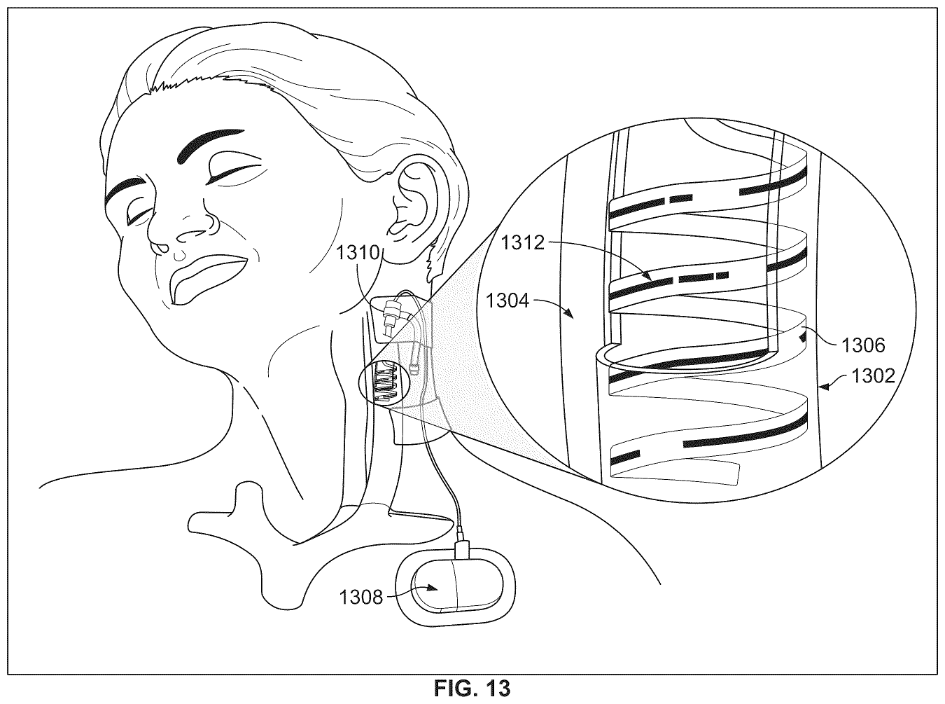

FIG. 13 is another graphic illustration of an exemplary implementation of the disclosed exemplary neural interface fibers. The components of the neuromodulation system 1200 may also be used in a system for placement of neural interface fiber 1306 in the internal jugular (IJ) vein 1302 adjacent to the vagus nerve 1304.

A controller, such as controller 1308, for example, which may be similar to controller 1102 of FIG. 11 or controller 1202 of FIG. 12, may be operable to output a signal that stimulates the vagus nerve 1304. In this exemplary implementation, the controller 1308 may be an implantable pulse generator (IPG), and at least part of the processing circuitry (described with reference to FIG. 11) may be part of the IPG. Alternatively, the controller 1308 may, for example, be a rechargeable, closed-loop external pulse generator (EPG), or the like. The neural interface fiber 1306 may be implanted in the IJ vein 1302 using, for example, an insertion device 1310, such as a central venus catheter or the like, and a customized deployment device. in the example, the controller 1308 may be operable to generate signals that are output from electrodes 1312 and operable to address migraines, depression, epilepsy, and/or other conditions treatable via electrical, chemical, and/or physical manipulation (e.g., temperature or pressure) of the vagus nerve 1304.

Although FIGS. 12 and 13 show the neural interface fiber, such as 1306 in FIG. 13, with a helical structure, the neural interface fiber may not be so limited and may have a shape or end suitable to implement the described functionality.

For example, the neural interface fiber may be drawn in a straight and linear structure, which may be utilized in very small diameter blood vessels. Additionally, other possible conformations of the fiber that we may employ include:
an arc structure;
a helix that doubles back on itself to create a 3D structure;
a pair of helices overlaid, one with right-handed configuration and the other with left-handed configuration;
a multi-filament splayed structure; and/or
a Flower structure or any structure with multiple filament loops.

For example, the neural interface fiber 1306 of FIG. 13 may have a straight configuration and be actuated into a helical shape during the implantation procedure. Exemplary methods to actuate the neural interface fiber into a helical structure inside a blood vessel may include: use of joule heating by passing a current through a high-resistance wire (e.g., an additional element or additional component) in the neural interface fiber, in combination with a thermally-responsive polymer near an outer surface of the fiber that may expand in response to the heating and cause the neural interface fiber to form a helix; or use of an “inflatable” microfluidic channel (e.g., an additional element or additional component) on a side of the fiber that expands upon delivery of fluid into the channel, causing that side of the fiber to expand and the fiber to form a helical shape. Of course, other examples of implementing a certain shape at an end of the neural interface fiber may be performed.

FIG. 14 illustrates an exemplary arrangement for the formation of a neural interface fiber according to the disclosed subject matter.

In the exemplary manufacturing environment 1400 in which an exemplary thermal fiber drawing process may be implemented is shown in FIG. 14, a preform 1402 may be produced at the macroscale and may be heated using, for example, a heating element 1404. Upon reaching a suitable temperature, the preform 1402 may be stretched by the application of force into an elongated fiber body 1406, which may be kilometers long, or the like. The elongated fiber body 1406 may also processed to have electrode features (e.g., openings that expose the conductive microwires 1408 therein) as shown in earlier examples.

In more detail with reference to the set up shown in FIG. 14, the conductive microwires 1408, also referred to as “conductive structures” herein, are manipulated to converge into the elongated fiber body 1406 during the drawing process. During the thermal fiber drawing process, a macroscale “preform” (i.e., preform 1402) composed, for example, of thermoplastic polymers, may be heated in a furnace, and a downward force (in the direction shown by the “downward force” arrow) via a pulling member or device is applied to stretch the preform 1402 into an elongated fiber body 1406 that may retain the same ribbon cross-sectional geometry as that of the preform 1402. Although the elongated fiber body 1406 resulting from the drawing process shown in FIG. 14 is shown to have a helical form, its depiction is merely to suggest one manner of storing a microscale fiber (e.g., in a helical form) after the drawing process, and is not meant to suggest the formation of a neural interface fiber having a helical shape.

In an example, the fabrication process for generating a usable neural interface fiber may involve using a preform 1402, for example, made of a polymeric material, and feeding electrically conductive microwires 1408. The electrically conductive microwires 1408 may include, for example, a carbon nanotube material or the like, and may be fed into a preform, such as 1402, as shown. Multiple conductive microwires 1408 may be longitudinally fed into the preform (parallel to the axis on which the neural interface fiber is being stretched, but in any pattern, such as those shown in FIG. 1 and FIG. 2. The elongated fiber body 1406 may have any cross-sectional geometry, such as a curved one (e.g., a circular cross section), or a polygonal one (e.g., as shown in the rectangular cross section of FIG. 2 and FIG. 3). Optionally, the elongated fiber bodies 1406 may include a microfluidic channel (such as additional element 112 of FIG. 1) to enable delivery of a fluid to a target site in a brain region of a subject.

Although the elongated fiber body 1406 is shown as having a ribbon cross-section, embodiments are not so limited, and include within their scope the formation of a microscale fiber (and resulting neural interface fibers) of any cross section, such as, for example a curved cross section (e.g., circular cross section, or oval cross section), or a polygonal cross section (e.g., a rectangular cross section as shown in FIG. 3 or substantially rectangular as shown in FIG. 2). The neural interface fiber may include electrodes at an outer peripheral region thereof (see e.g., FIGS. 4-10C), for example electrodes spaced, in a cross-sectional view of the neural interface fiber, in any manner, such as, for example, equidistantly with respect to one another.

In an example, the cladding 1410 of an elongated fiber body 1406 may be a dielectric insulation having material properties that include a relatively low water absorption rate, a relatively high dielectric constant, a relatively low Young's modulus, and biocompatibility. Reference is made to Table 1 below, which shows some dielectric materials suitable for use as the cladding 1410 of the elongated fiber body 1406 according to some embodiments.

Some embodiments include within their scope the use of other dielectric materials not shown in Table 1. For example, some embodiments include within their scope the use of a dielectric material for the cladding 1410 which may have any of the water absorption percentage ranges shown in Table 1, combined with any of the dielectric constant ranges shown in Table 1, and also combined with any of the Young's modulus ranges shown below.

In addition, some embodiments of a cladding 1410, which may have:
a water absorption percentage within a range starting at a lowest value as between the water absorption percent ranges noted in Table 1, and up to and including a highest value of the water absorption percent ranges noted in Table 1;
a dielectric constant within a range starting at a lowest value as between the water absorption ranges noted in Table 1, and up to and including a highest value of the water absorption ranges noted in Table 1; and/or
a Young's modulus within a range starting at a lowest value as between the Young's modulus ranges noted in Table 1, and up to and including a highest value of the Young's modulus ranges noted in Table 1.

TABLE 1

| | Water | Dielectric | Young's | |
|---|---|---|---|---|
| Polymer | Absorption | constant | modulus | |
| Polycarbonate | 0.12-0.35% | 2.7-3.17 | 1.58-3.24 | GPA |
| (PC) | | | | |
| Polyetherimide | 0.0-1.3% | 3.44 @60 Hz | 0.00280-56.0 | GPa |
| (PEI) | | 2.52-6.80 | | |
| Liquid Crystallin | 0-0.04% | 3.16 | 7-23.5 | GPa |
| Polymer (LCP) | | Loss tangent | | |
| | | 0.0049 | | |
| | | (measured | | |
| | | at 30 GHz) | | |
| Polyphenol | 0.2-1.2% | 3.44 @60 Hz | 0.0689-8.14 | GPa |
| sulfone (PPSU) | | 3.00-3.90 | | |
| Thermoplastic | 0.2-1.3% | 3.90-6.50 | 0.005-1.9 | GPa |
| Polyurethane | | | | |
| (TPU | | | | |
| Poly- | 0.2-0.8% | 3.8 @50 Hz | 2.7 | GPa |
| oxymethylene | | | | |
| (POM) | | | | |
| Polymethyl- | 0.1-0.8% | 2.5-3.3 | 1.80-3.1 | GPa |
| methacrylate | | | | |
| (PMMA) | | | | |
| Styrene-Ethylene- | 0.2-0.4% | 2.2 @ 1 KHz | 6 | MPa |
| Butylene-Styrene | | | | |
| (SEBS) | | | | |
| Polyimide (PI) | 0.3-1.43 | 3.0-3.7 | 1.3-4 | GPa |
| | | @1 MHz | | |

The dielectric material of the cladding 1410 may, by way of example, include one or more of the polymers listed in Table 2 below, the noted polymers having the respective glass transition temperatures in degrees Celsius (Tg° C.), and the respective annealing temperatures (with the noted error margin of about +35° C. to +50° C.) as shown in Table 2 in which the annealing pertains to one way of forming a helical structure for the elongated fiber body 1406. A neural interface fiber may be formed into a helix, by way of example using annealing, as will be described in further detail in relation to FIG. 15A to FIG. 15C. Such a process may include the annealing temperatures and annealing durations as noted in Table 2 when the cladding 1410 is made of any one or more of the corresponding materials listed by way of example below.

TABLE 2

| | | Annealing | |
|---|---|---|---|
| | | Temperature | |
| | Tg | (~Tg + | Duration of |
| Polymer | (° C.) | 35-50° C.) | annealing |
| Polycarbonate (PC) | 147 | 185 | ~5 min-1 hr |
| Polyetherimide (PEI) | 215-220 | 260 | ~5 min-1 hr |
| Liquid Crystallin | 110-130 | 160 | ~5 min-1 hr |
| Polymer (LCP) | | | |
| Polyphenolsulfone | 220-230 | 260 | ~5 min-1 hr |
| (PPSU) | | | |
| Thermoplastic | −50-100 | 100 | ~5 min-1 hr |
| Polyurethane (TPU | | | |
| Polyoxymethylene | −60-80 | 120 | ~5 min-1 hr |
| (POM) | | | |
| Polymethylmethacrylate | 105 | 140 | ~5 min-1 hr |
| (PMMA) | | | |
| Styrene-Ethylene- | −60-100 C. | 130 | ~5 min-1 hr |
| Butylene-Styrene | | | |
| (SEBS) | | | |
| Polyimide (PI) | 300 C. | 350 | ~5 min-1 hr |

For example, the electrically conductive elements or electrically conductive structures, i.e., conductive microwires 1408, of an elongated fiber bodies 1406 may include carbon nanotube (CNT) microwires, platinum, or tungsten. Additionally, the electrically conductive elements or electrically conductive structures may be formed using electrically conductive material(s).

As used herein, “electrically conductive material” includes an electrically conductive material such as a metal (e.g., copper, aluminum, nickel, cobalt, iron, tin, gold, silver, platinum, platinum-iridium, tungsten, stainless steel, or combinations thereof) or an organic material such as a material including carbon, or a polymer. For example, when fabricating the electrically conductive elements or electrically conductive structures, electrically conductive filaments may be coated with an additional layer of dielectric material prior to thermal fiber drawing to enhance the conductive element's or structure's electrical insulation, to improve its biocompatibility, and/or to modify its geometry to improve compatibility with the fiber drawing process and/or machinery. This process, for example, is beneficial to make irregularly-shaped carbon wires used for the conductive structures rounder and more regular.

In a further example, the hypothetical manufacturing environment 1400 may be configured to utilize a dissolvable hydrogel coating along with pre-annealing the elongated fiber body 1406 into a helical shape. In this scenario, the preform 1402 is thermally-annealed as described above, then as an elongated fiber body 1406 with a helical structure, the elongated fiber body 1406 is coated with a dissolvable hydrogel (such as polyethylene glycol (PEG) or the like), and dried in a straight configuration as a neural interface fiber ready for use in neuromodulation treatments. The dried hydrogel causes the elongated fiber body 1406 to retain a straight configuration. When the neural interface fiber, such as neural interface fiber 700, is delivered into a blood vessel as part of a neuromodulation treatment, the hydrogel will hydrate and dissolve, in this way releasing the helical structure set as the elongated fiber body 1406, to return back into its pre-set helical configuration.

Alternatively, or in addition, the hypothetical manufacturing environment 1400 may include a shape memory material (not shown in this example) that is combined with the preform 1402 during the drawing process to form the elongated fiber body 1406. The shape memory material may be preset to a desired helical shape at one temperature. However, in some instances, the drawing temperature is at a temperature in the range of the deformable phase for the shape memory material. The shape memory material may be embedded in the neural interface fiber body during drawing while it is straight and remain straight until implantation into a vessel of a subject, at which time it may be heated to a body temperature and assume its helical shape again. In the example, the preform 1402 is drawn into a 1406 fiber body. To form the helix, the elongated fiber body 1406 is annealed into a helical shape. The elongated fiber body 1406 is straightened, and coated with a dissolvable hydrogel, which is dried while the elongated fiber body 1406 is in a substantially straight configuration. Upon insertion in the blood vessel, or the tissue, the dissolvable hydrogel will dissolve, and the elongated fiber body 1406 returns to its helical shape. In a further example, an additional layer of material may be added to the cladding of a neural interface fiber to enhance blood compatibility and reduce thrombosis (i.e., blood clot) risk. This material could be drawn with the fiber during the fiber drawing process by incorporating it as a layer on the outside of the preform 1402, or it could be added after the elongated fiber body 1406 is formed via thermal drawing. If the layer is added after the elongated fiber body 1406 is formed, it could be added by, for example, dip-coating or spray coating the layer onto the elongated fiber body 1406 or by co-extrusion around the fiber.

When finishing the fabrication of the neural interface fibers, a helical structure may be formed prior to cutting the elongated fiber body 1406 into the number of neural interface fibers. Alternatively, when finishing the fabrication of the neural interface fibers, a helical structure may be formed after cutting the elongated fiber body 1406 into the number of neural interface fibers.

In a summary, a neural interface fiber according to some embodiments may be formed by way of thermal drawing, which may be used to draw macroscale machined fiber preforms into microscale implantable fibers, with spooled CNT or other conductive structures incorporated during the drawing procedure through convergence. The drawing procedure may produce tens to hundreds of meters of flexible fiber, with functional capabilities accessible by connectorizing individual elements with input/output features such as optical ferrules, electrical pins, and microfluidic tubing. Fiber devices may be used to interface with a single brain region or, due to the small footprint of the implanted portion, combined into devices for dual-site access, with each individual fiber remaining capable of performing each of multiple desired functions.

An example of a process to form a portion of the elongated fiber body 1406 into a helical structure is described in more detail with reference to the examples of FIGS. 15A-15C.

FIG. 15A illustrates a perspective view of an exemplary system component usable in a larger system to form an exemplary helical structure of an exemplary neural interface fiber according to the disclosed subject matter.

The process described with reference to FIG. 15A-FIG. FIG. 15C provides a helical structure prior to implantation of the neural interface fiber inside vessels of a subject.

According to the example of FIGS. 15A-15C, a mold in the shape of a rigid mandrel 1502, may be provided that defines helical therein. The groove 1504 is configured to receive a “straight” (e.g., non-helical) neural interface fiber therein along a length of the mandrel 1502 in a helical (or spiral) fashion, and the assembly is annealed to yield a neural interface fiber having a helical structure. The grooves 1504 may, as shown in cross-section cross section 1508, have a configuration to complement a configuration of a cross section of the neural interface fiber. For example, in the case shown in FIGS. 15A-15C, the grooves 1504 may have an open rectangular cross-sectional configuration with a width X and a height Z substantially matching a width and a height of the neural interface fiber to be received therein. Thus, mandrel 1502 accommodates a neural interface fiber having a ribbon-like structure, such as that shown and described with reference to the earlier examples above. In another embodiment, for example, where the neural interface fiber has a circular configuration, the groove 1504 may, for example, correspond to a semicircular concave configuration in a cross section 1508 of the mandrel 1502. The cross section 1508 may have a diameter of the associated semicircle corresponding substantially to a diameter of the neural interface fiber.

FIG. 15B illustrates a side view of an exemplary system component usable in a larger system to form an exemplary helical structure of an exemplary neural interface fiber according to the disclosed subject matter.

Mandrel 1502 in the shown embodiment has a proximal end 1506a and a distal end 1506b. In the shown example of FIG. 15B, a helical pitch of the groove 1504 may increase in a direction from the proximal end 1506a to the distal end 1506b, in this way resulting in neural interface fiber having a similar helical structure, as suggested in the assembly 1710 of FIG. 15C in particular, where a portion of the neural interface fiber 1514 is depicted as having been disengaged from the mandrel 1502 after formation thereof.

According to an embodiment, a range of potential pitch/spacing between turns of the turns of the helical structure of a neural interface fiber may be between about 25 microns to about 1 m, or the like.

FIG. 15C illustrates a perspective view of an exemplary helical structure of an exemplary neural interface fiber disengaged from an exemplary system component after formation of helical structure according to the disclosed subject matter.

Alternate methods and processes for fabricating the neural interface fiber are also contemplated.

For example, a first alternate method of providing a neural interface fiber having a helical structure may include strain engineering, during which a combination of an elastomer component and a non-elastomer component may be co-drawn together to form the fiber as shown in FIG. 16 and FIGS. 15A-15C with an elastomer retaining linear stresses from the draw. Thereafter, the non-elastomer sacrificial component may be removed, in this way releasing the elastomer component, which would retract, causing formation of a helix.

A second alternate method of providing a neural interface fiber having a helical structure may include the use of a shape memory material embedded in the cladding, such as a shape memory alloy or a shape memory polymer. A shape memory alloy to be embedded in the cladding for the formation of a helical structure according to some embodiments may include Nitinol. Another suitable shape memory alloy may include a copper-aluminum-nickel shape memory alloy. One or more shape memory polymers to be embedded in the cladding for the formation of a helical structure may include polyurethane, polyesterester, polyesterurethane, polyester, poly (¿-caprolactone)/poly (butylene terephthalate), it being noted a “shape memory polymer” may have shape memory characteristics by virtue of its composition, the manufacturing process for the formation of the same, the presence of one or more dopants therein.

In an example, the shape of the shape memory portion may be preset into a helical shape, for example, at high temperatures. Nitinol is an example of a shape memory alloy that may be used and may be preset in a shape by applying heat at about 500 degrees Celsius. The Nitinol may then be cooled. During drawing of the fiber using heat, the shape memory portion may assume a linear shape along a length thereof when it is incorporated into the fiber body (see e.g., FIGS. 2, 3 and 14). When the neural interface fiber is cooled, the shape memory portion reverts to its preset helical shape.

According to some embodiments, the provision of electrodes, including any additional processing thereof, such as the addition of layers, the provision of radiopaque markers and end plugs may be performed after the drawing process (see e.g., FIG. 14-FIG. 15C) and before or after a shaping of the resulting structure into a helical form if a helical form is desired for the neural interface fiber.

In an example, a neural interface fiber according to an embodiment may include a cladding and electrically conductive structures therein, exposed portions of the electrically conductive structures corresponding to electrodes of the neural interface fiber, the neural interface fiber having a helical structure.

A neural interface fiber according to an embodiment may include a cladding and electrically conductive structures therein, exposed portions of the electrically conductive structures corresponding to electrodes of the neural interface fiber, the neural interface fiber having a helical structure, the neural interface fiber part of a neural interface fiber assembly that includes an expansion structure at a helical distal end of the neural interface fiber.

A neural interface fiber according to an embodiment may include a cladding and electrically conductive structures therein, exposed portions of the electrically conductive structures corresponding to electrodes of the neural interface fiber, the neural interface fiber having a helical structure, the neural interface fiber part of a neural interface fiber assembly that includes a microcatheter within which the neural interface fiber is housed for delivery.

A neural interface fiber according to an embodiment may include a cladding and electrically conductive structures therein, exposed portions of the electrically conductive structures corresponding to electrodes of the neural interface fiber, the electrodes within recesses in the neural interface fiber structure.

A neural interface fiber according to an embodiment may include a cladding and electrically conductive structures therein, exposed portions of the electrically conductive structures corresponding to electrodes of the neural interface fiber, the electrodes protruding from an outer surface of the neural interface fiber.

A neural interface fiber according to an embodiment may include a cladding and electrically conductive structures therein, exposed portions of the electrically conductive structures corresponding to electrodes of the neural interface fiber. The neural interface fiber may have a helical structure. The electrodes may have a strip configuration, and individual ones of the electrodes may span one of ¼ or ½ of a perimeter of a corresponding loop of the helical structure, by way of example.

A neural interface fiber according to an embodiment may include electrodes arranged randomly across a surface thereof.

A neural interface fiber according to an embodiment may include a cladding and electrically conductive structures therein, exposed portions of the electrically conductive structures corresponding to electrodes of the neural interface fiber, the neural interface fiber having a helical structure, the exposed portions of the electrically conductive structures including additional layers of conductive material thereon.

A neural interface fiber according to an embodiment may include a cladding and electrically conductive structures therein, exposed portions of the electrically conductive structures corresponding to electrodes of the neural interface fiber, the neural interface fiber having a helical structure, the exposed portions of the electrically conductive structures including layers of dielectric surrounding the same.

A neural interface fiber according to an embodiment may include a cladding and electrically conductive structures therein, exposed portions of the electrically conductive structures corresponding to electrodes of the neural interface fiber, the neural interface fiber having a helical structure, and the electrodes having a strip configuration, the neural interface fiber including radio-opaque markers thereon at a distal surface of the neural interface fiber.

A neural interface fiber according to an embodiment may include a cladding and electrically conductive structures therein, exposed portions of the electrically conductive structures corresponding to electrodes of the neural interface fiber, the neural interface fiber having a helical structure, and the electrodes having a strip configuration, the cladding including a polymer material having polymer chains that are substantially non-linearly arranged with respect to one another.

A neural interface fiber according to an embodiment may include a cladding and electrically conductive structures therein, exposed portions of the electrically conductive structures corresponding to electrodes of the neural interface fiber, the neural interface fiber having a helical structure, a distant end of the neural interface fiber having a geometry and/or stiffness to allow the neural interface fiber to pierce through a blood vessel.

Advantageously, some embodiments of neural interface fiber disclosed herein may be deployed into vessels throughout the brain and body to record and modulate activity in the brain and peripheral nervous system. Due to the hair-thin fiber form-factor and high flexibility of the neural interface fiber, the device can navigate tortuous vasculature and reach small (<1 mm diameter) vessels to serve as a neural interface at diverse targets throughout the brain and body which are challenging to reach with other technologies.

Advantageously, the hair-thin fiber form-factor and high flexibility of the exemplary neural interface fiber described herein allows it to be placed, for example, within a standard catheter and be integrated with existing surgical workflows in cardiac and neuro-vascular catheterization laboratories to be deployed in target vessels across the body.

Advantageously, a neural interface fiber according to some embodiments may also be compatible with MRI, enabling its use for correlating local recording or manipulations of circuit function to brain-wide mapping of neural states.

Advantageously, a neural interface fiber may have a cladding that may be flexible and biocompatible, enabling its use as an implant for chronic long-term use (e.g., greater than 6 months).

Advantageously, an neural interface fiber according to some embodiments may be configured for the recording of and modulation of the activity of neurons, nerve bundles, glia, and/or other organ systems, and it may be used as an investigational research tool in the study of neuroscience, neuromuscular physiology and disorders, as well as a clinical tool for diagnostics, monitoring, and therapeutic interventions.

Advantageously, a neural interface fiber according to some embodiments may also have a magnetic susceptibility close to that of water and biological tissues (e.g., Xm of about-10 ppm) making it suitable for use with MRI.

Advantageously, an neural interface fiber according to some embodiments may include electrodes, such as electrodes including CNT, with high surface areas through nano- or microscale roughness or porosity, which may reduce the electrode impedance, increase the CIC for electrical stimulation, and facilitate the adhesion of neurochemicals to increase the sensitivity of any FSCV measurements.

Advantageously, a neural interface fiber according to some embodiments provides a platform to study the brain, muscles, nerves, and other organ systems across several modalities and has several different applications in research.

Advantageously, a neural interface fiber according to some embodiments may be used to deliver patterned electrical stimulation along the length of the neural interface fiber to provide versatility in the spatial extent of stimulation.

Advantageously, a neural interface fiber according to some embodiments may be used for the diagnosis and treatment of drug-resistant epilepsies. The neural interface fiber may be used to localize seizure onset zones and ablate pathologic tissue to prevent seizure onset. This may be achieved using the neural interface fiber for electrophysiology recording in the brain (e.g., as a stereo EEG electrode) while also locally delivering drugs to transiently inhibit brain regions and observing the effects on the seizure onset network. After localizing the seizure onset zone via electrophysiology and drug delivery, targeted ablation may be performed with the neural interface fiber through coupling a CO2 laser to the optical waveguide, delivering electrical or radiofrequency (RF) ablation, and/or chemical and/or pharmacological ablation by delivery of drug payloads through the microfluidic channel.

Advantageously, a neural interface fiber according to some embodiments may enable, the concurrent recording and modulation of six techniques including: (1) electrophysiology, (2) electrical stimulation, (3) optical recording via fiber photometry, (4) optogenetic stimulation,, and (6) local delivery of drug and gene payloads through a fluidic channel. By combining these techniques into a single neural interface fiber, a neural interface fiber according to some embodiments disclosed herein may allow for the probing of the interplay between electrophysiological and neurochemical signaling during behavior, as well the studying of the neural responses to deep brain stimulation (DBS), a technique that is widely used clinically for treating the motor symptoms of Parkinson's disease as well as a number of other disorders, but whose mechanism of action in the brain remains poorly understood. Furthermore, the neural interface fiber disclosed herein may enable the local delivery of drugs or genetic payloads while concurrently recording their effect on neural activity. Finally, the neural interface fiber disclosed herein may function in the MRI environment, allowing the local neural circuit recording and modulation techniques to be linked to whole-brain activity during MRI.

Advantageously, a neural interface fiber according to some embodiments may be utilized for a variety of applications in research and healthcare. For example, they may be leveraged for basic neuroscience research aimed at investigating neural dynamics and exploring the effects of neuromodulation. A neural interface fiber may also be used as a medical device, with potential applications including but not limited to, distributed electromyography (EMG) recording in muscles for prosthetics interfacing and rehabilitation medicine, stimulating in muscle to prevent muscle atrophy after injury or stroke, local drug delivery into the brain, muscles, or peripheral organs, recording and stimulation in the brain for neurodiagnostics and monitoring, and/or brain computer interfaces.

Advantageously, a neural interface fiber according to some embodiments may be produced in a scalable thermal drawing process, where macroscopic models (preforms) of the target neural interface fiber are heated and drawn into tens to hundreds of meters of micro structured fibers with conserved cross-section geometrically identical to those of the preforms. This enables, advantageously, fabrication of variable length endovascular devices (i.e. length can be tailored to patient anatomy and vessel target) with embedded electrodes which can be connected to a control system after exiting through a vessel wall, without the need for any interface between the embedded microwires to be formed inside of the vessel. Furthermore, the components of the neural interface fiber are drawn from generally biocompatible materials and fabrication techniques.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain, using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Although an overview of embodiments has been described with reference to specific example embodiments, various modifications and changes may be made to these embodiments without departing from the broader scope of embodiments of the present disclosure. Such embodiments of the inventive subject matter may be referred to herein, individually or collectively, by the term “invention” merely for convenience and without intending to voluntarily limit the scope of this application to any single disclosure or inventive concept if more than one is, in fact, disclosed.

The embodiments illustrated herein are described in sufficient detail to enable those skilled in the art to practice the teachings disclosed. Other embodiments may be used and derived therefrom, such that structural and logical substitutions and changes may be made without departing from the scope of this disclosure. The Detailed Description, therefore, is not to be taken in a limiting sense, and the scope of various embodiments is defined only by the appended claims, along with the full range of equivalents to which such claims are entitled.

It will also be understood that, although the terms “first,” “second,” and so forth may be used herein to describe various elements, these elements should not be limited by these terms. These terms are only used to distinguish one element from another. For example, a first contact could be termed a second contact, and, similarly, a second contact could be termed a first contact, without departing from the scope of the present example embodiments. The first contact and the second contact are both contacts, but they are not the same contact.

As used in the description of the example embodiments and the appended examples, the singular forms “a,” “an,” and “the” are intended to include the plural forms as well, unless the context clearly indicates otherwise. It will also be understood that the term “and/or” as used herein refers to and encompasses all combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B, and C).

In embodiments, the phrase “A is located on B” means that at least a part of A is in direct physical contact or indirect physical contact (having one or more other features between A and B) with at least a part of B.

In the instant description, “A is adjacent to B” means that at least part of A is in direct physical contact with at least a part of B.

In the instant description, “A is attached to B” means that at least part of A is mechanically attached to at least part of B, either directly or indirectly (having one or more other features between A and B).

In the instant description, “the As are coupled to the Bs” means that at least some of the As are coupled to at least some of the Bs, and not necessarily that all As are coupled to at least one B and all Bs are coupled to at least one A.

In the instant description, “A is within B” means that at least some of A is encompassed within the physical boundaries of B.

The use of reference numerals separated by a “/”, such as “102/104” for example, is intended to refer to 102 or 104 as appropriate. Otherwise, the forward slash (“/”) as used herein means “and/or.”

When used to describe a range of dimensions, the phrase “between X and Y” represents a range that includes X and Y.

Although certain elements may be referred to in the singular herein, such elements may include multiple sub-elements. For example, “an insulating material” may include one or more insulating materials. As used herein, a “conductive contact” may refer to a portion of conductive material (e.g., metal) serving as an electrical interface between different components; conductive contacts may be recessed in, flush with, or extending away from a surface of a component, and may take any suitable form (e.g., a conductive pad or socket, or portion of a conductive line or via).

The use of the techniques and structures provided herein may in some instances be detected using tools such as: electron microscopy including scanning/transmission electron microscopy (SEM/TEM), scanning transmission electron microscopy (STEM), nano-beam electron diffraction (NBD or NBED), and reflection electron microscopy (REM); composition mapping; x-ray crystallography or diffraction (XRD); energy-dispersive x-ray spectroscopy (EDX); secondary ion mass spectrometry (SIMS); time-of-flight SIMS (ToF-SIMS); atom neural interface fiber imaging or tomography; local electrode atom neural interface fiber (LEAP) techniques; 3D tomography; or high resolution physical or chemical analysis, to name a few suitable example analytical tools. Such tools can indicate an integrated circuit including at least one semiconductor package including an embedded magnetic inductor.

In some embodiments, the techniques, processes and/or methods described herein can be detected based on the structures formed therefrom. In addition, in some embodiments, the techniques and structures described herein can be detected based on the benefits derived therefrom. Numerous configurations and variations will be apparent considering this disclosure.

The description may use perspective-based descriptions such as top/bottom, in/out, over/under, and the like. Such descriptions are merely used to facilitate the discussion and are not intended to restrict the application of embodiments described herein to any particular orientation.

The description may use the phrases “in an example,” “according to some examples,” “in accordance with embodiments,” or “in examples,” which may each refer to one or more of the same or different examples. Furthermore, the terms “comprising,” “including,” “having,” and the like, as used with respect to embodiments of the present disclosure, are synonymous.

“Coupled” as used herein means that two or more elements are in direct physical contact, or that that two or more elements indirectly physically contact each other, but yet still cooperate or interact with each other (i.e., one or more other elements are coupled or connected between the elements that are said to be coupled with each other). The term “directly coupled” means that two or more elements are in direct contact.

In the corresponding drawings of the embodiments, signals, currents, electrical biases, or magnetic or electrical polarities may be represented with lines. Some lines may be thicker, to indicate more constituent signal paths, and/or have arrows at one or more ends, to indicate primary information flow direction. Such indications are not intended to be limiting. Rather, the lines are used in connection with one or more exemplary embodiments to facilitate easier understanding of a circuit or a logical unit. Any represented signal, polarity, current, voltage, etc., as dictated by design needs or preferences, may actually comprise one or more signals that may travel in either direction or may be implemented with any suitable type of signal scheme.

Throughout the specification, and in the claims, the terms “coupled” or “connected” mean a direct or indirect connection, such as a direct electrical, mechanical, or magnetic connection between the elements that are connected or an indirect connection, through one or more passive or active intermediary devices.

The term “signal” may refer to at least one current signal, voltage signal, magnetic signal, or data/clock signal.

The meaning of “a,” “an,” and “the” include plural references.

The meaning of “in” includes “in” and “on.”

The terms “substantially,” “close,” “approximately,” “near,” and “about,” generally refer to being within +/−10% of a target value (unless specifically specified). Unless otherwise specified the use of the ordinal adjectives “first,” “second,” and “third,” etc., to describe a common object, merely indicate that different instances of like objects are being referred to, and are not intended to imply that the objects so described must be in a given sequence, either temporally, spatially, in ranking or in any other manner, and are not intended to imply that the objects so described must necessarily be made of different materials or have different dimension.

What is claimed is:

1. A neural interface fiber, comprising:
a plurality of conductive structures;
a cladding adapted to substantially encompass the plurality of conductive structures, wherein the cladding is a dielectric insulation;
wherein the cladding includes one or more openings exposing a portion of a respective conductive structure of the plurality of conductive structures within the cladding enabling the exposed portion to contact tissue external to the cladding at the one or more openings; and
an end cap fastened to a distal end of the cladding and configured to electrically insulate the plurality of conductive structures, wherein the plurality of conductive structures are configured to connect to an electrical signaling/recording device.

2. The neural interface fiber of claim 1, wherein the conductive structure comprises conductive microwires.

3. The neural interface fiber of claim 2, wherein the conductive microwire comprises carbon-based filament, platinum, tungsten, organic material including conducting polymers, doped polymers, carbon-polymer blends, platinum iridium (PtIr), stainless steel, gold, any metal, non-metal conducting microwire, or combinations thereof.

4. The neural interface fiber of claim 3, wherein the carbon-based filament has a low electrochemical impedance, a high charge storage capacity, and a high charge injection capacity.

5. The neural interface fiber of claim 1, wherein each conductive structure of the plurality of conductive structures is operable to conduct stimulation signals generated by the electrical signaling/recording device to the tissue without supplementation.

6. The neural interface fiber of claim 1, wherein the respective conductive structures are operable to carry signals generated by the electrical signaling/recording device an entire length of the respective conductive structure from the electrical signaling/recording device to the end cap.

7. The neural interface fiber of claim 1, wherein the exposed portion of the respective conductive structures are operable to deliver electrical impulses provided by the electrical signaling/recording device to stimulate tissues through a blood vessel wall.

8. The neural interface fiber of claim 1, wherein the exposed portions of the respective conductive structures are operable to enable recording responses to stimulation of tissues through a blood vessel wall provided by the electrical signaling/recording device.

9. The neural interface fiber of claim 1, further comprising:
a layer or a film configured to enhance blood compatibility and/or reduce thrombosis risk on or near an exterior of the cladding.

10. The neural interface fiber of claim 1, wherein the end cap comprises:
a hole or loop configured to couple to a guidewire or delivery mechanism, wherein the releasable hole or loop enables the neural interface fiber to be pushed to a location within a blood vessel and be released.

11. The neural interface fiber of claim 1, wherein the end cap comprises:
a radiopaque material operable to enable the end cap to be visible via an imaging device when the end cap is within a blood vessel.

12. The neural interface fiber of claim 1, wherein the cladding further comprises:
a plurality of radiopaque markers along a portion of a length of the cladding adjacent to the end cap for a predetermined length, wherein each of the radiopaque markers is operable to enable a portion of the cladding to be visible via an imaging device when the cladding is within a blood vessel.

13. The neural interface fiber of claim 1, wherein the cladding further comprises:
a plurality of radiopaque markers along an entire length of the cladding, wherein each of the radiopaque markers is operable to enable a portion of the cladding to be visible via an imaging device when the cladding is within a blood vessel.

14. The neural interface fiber of claim 1, further comprising:
a polymer composite within the cladding, wherein the polymer composite is configured to be adjacent to one or more of the plurality of conductive structures for a length of the respective one or more of the plurality of conductive structures within the neural interface fiber.

15. The neural interface fiber of claim 14, wherein the polymer composite comprises:
tungsten powder; and
an elastomer matrix, wherein the load density of the tungsten powder with respect to the elastomer matrix is sufficiently high to enable radiopacity of the polymer composition and compatibility with thermal fiber drawing manufacturing of the neural interface fiber.

16. The neural interface fiber of claim 1, wherein:
a portion of the neural interface fiber has a helical structure originating at the end cap and extending a predetermined length from the end cap.

17. The neural interface fiber of claim 1, further comprising:
a helical structure of the plurality of respective conductive structures and cladding, wherein a portion of the neural interface fiber having the helical structure, when within a blood vessel, is operable to place the one or more exposed portions of the respective conductive structures in contact with an inner wall of the blood vessel to enable stimulation of tissues and recording of stimulation response activity through the vessel wall by the electrical signaling/recording device.

18. The neural interface fiber of claim 1, wherein the cladding is a thermoplastic polymer having annealing properties that enable the cladding to be annealed into a helical structure.

19. The neural interface fiber of claim 1, wherein the helical structure at the distal end of the cladding is configured to enclose an expansion structure, wherein the expansion structure is operable, upon release, to expand to press the exposed portion to facilitate contact between the exposed portion and a blood vessel wall.

20. The neural interface fiber of claim 1, wherein the helical structure comprises:
a loop configuration, a double helix configuration, or a right-handed helix overlaid with a left-handed helix configuration, wherein any of the configurations is operable to facilitate contact between the exposed portion and a blood vessel wall.

21. The neural interface fiber of claim 1, wherein the cladding has a substantially rectangular or ribbon cross-section configured with rounded corners.

22. The neural interface fiber of claim 1, further comprising:
a shape memory portion, wherein the shape memory portion extends along a length of the cladding and is positioned adjacent to the conductive structures.

23. A neural interface fiber, comprising:
a plurality of conductive structures; and
a cladding adapted to insulate the plurality of the conductive structures, wherein exposed portions of respective conductive structures of the plurality of conductive structures in the cladding are electrodes operable to contact tissue external to the cladding.

24. The neural interface fiber of claim 23, wherein the plurality of conductive structures further comprises:
termination points at a proximal end of the plurality of conductive structures and operable to couple to an electrical signaling/recording device.

25. The neural interface fiber of claim 24, wherein each respective termination point is an end point of a respective conductive structure of the plurality of conductive structures.

26. The neural interface fiber of claim 23, wherein the plurality of conductive structures further comprises:
termination points providing access to the plurality of conductive structures through the cladding at one or more locations along the length of the neural interface fiber, wherein the termination points are operable to couple to an electrical signaling/recording device.

27. The neural interface fiber of claim 23, wherein the plurality of conductive structures further comprises:
termination points at a distal end of the plurality of conductive structures and operable to output a stimulation signal and/or receive a feedback signal.

28. The neural interface fiber of claim 24, wherein each respective termination point at the distal end is an end point of a respective conductive structure of the plurality of conductive structures.
